(19) United States
(12) Patent Application Publication
(10) Pub. No.: US 2022/0205033 A1
(43) Pub. Date: Jun. 30, 2022

(54) System and Method for Control of Sequencing Process

(71) Applicant: LIFE TECHNOLOGIES CORPORATION, Carlsbad, CA (US)

(72) Inventors: Mark REED, Menlo Park, CA (US); Joseph PODHASKY, San Rafael, CA (US); Shanti SHANKAR, Cheshire, CT (US)

(21) Appl. No.: 17/553,295

(22) Filed: Dec. 16, 2021

Related U.S. Application Data
(60) Provisional application No. 63/132,479, filed on Dec. 31, 2020.

(51) Int. Cl. C12Q 1/6869; C12Q 1/6806; G16B 50/20

(52) U.S. Cl. CPC C12Q 1/6869; G16B 50/20; C12Q 1/6806

(57) ABSTRACT

A method for determining a sequence of nucleic acids includes purifying the nucleic acids from a sample with a purification instrument in accordance with a purification plan of a run plan. The purified nucleic acids are disposed in a transfer plate. Disposition of the purified nucleic acids is stored in a transfer file. The method further includes transferring the transfer plate to a sequencing instrument; transferring the transfer file to the sequencing instrument; and sequencing the nucleic acids with the sequencing instrument in accordance with a sequencing plan of the run plan and based on the transfer file.

### CROSS-REFERENCE TO RELATED APPLICATION(S)

This application claims benefit of U.S. Provisional Application No. 63/132,479 filed Dec. 31, 2020, which is incorporated herein by reference in its entirety.

### BACKGROUND

Increasingly, genetic sequencing is being used as a tool in both research and clinical settings. For example, research into the origins of disease, differentiations of species, characteristics of microbiomes, and the study of both bacterial and viral pathogens is being performed using genetic sequencing. In another example, genetic testing is increasingly being used to detect cancers, trace viral infections, prescribe diets, and modify prescription formularies.

With the increased interest in use of genetic sequencing, demand has risen for automated solutions. Generally, nucleic acids are extracted from sources and purified. The purified nucleic acids are then sequenced.

Nucleic acid can be extracted from many sources using different techniques. For example, techniques for extraction of Formalin Fixed Paraffin Embedded (FFPE) samples, biopsies, or blood sources, among others. In particular, cell free DNA recovered from blood or plasma is increasingly becoming of interest. Moreover, extracting nucleic acids, such as DNA or RNA, from a plurality of samples simultaneously is of interest, particularly in clinical settings.

Once extracted, various techniques can be used to sequence the nucleic acid and analyze the results. Analysis may include detecting variants indicative of a condition or disease or a sensitivity to a medication. The use of sequencing can be limited by assay availability, sequencing run time, preparation time, and cost. Additionally, quality sequencing has historically been an expensive process, thus limiting its practice.

As such an improved sequencing system would be desirable.

### BRIEF DESCRIPTION OF THE DRAWINGS

The present disclosure may be better understood, and its numerous features and advantages made apparent to those skilled in the art by referencing the accompanying drawings.

FIG. 1 includes a flow diagram illustrating an example method.

FIG. 2 includes an illustration of an example sequencing system.

FIG. 3 includes an illustration of an example sequencing instrument.

FIG. 4 includes an illustration of an example purification instrument.

FIG. 5, FIG. 6, and FIG. 7 include block flow diagrams of example methods for performing a sequencing run.

FIG. 8 includes an illustration of an example purification instrument.

FIG. 9 and FIG. 10 include illustrations of an example platform of a purification instrument.

FIG. 11 and FIG. 12 include illustrations of example transfer and archive trays.

FIG. 13 includes an illustration of an example sequencing instrument.

FIG. 14 includes an illustration of an example deck of a sequencing instrument.

FIG. 15 includes an illustration of bulk reagent storage for a sequencing instrument.

FIG. 16 includes a schematic diagram of server system components.

FIG. 17 includes a block diagram of the analysis pipeline.

FIG. 18 includes a schematic diagram of generating an assay definition file.

FIG. 19 includes a schematic diagram of an example of the assay definition file packaging.

FIG. 20 includes an illustration of an example sequencing system.

FIG. 21 includes an illustration of an example system including a sensor array.

FIG. 22 includes an illustration of an example sensor and associated well.

FIG. 23 includes an illustration of an example method for preparing a sequencing device.

FIG. 24 includes an illustration of schema for seeding a support.

The use of the same reference symbols in different drawings indicates similar or identical items.

### DETAILED DESCRIPTION

In an embodiment, a system for sequencing nucleic acids derived from samples includes a purification instrument, a sequencer, and a server. Optionally, the sequencer and the server can reside in the same instrument, such as a sequencing instrument. The purification instrument can extract and isolate nucleic acids from samples such as environmental samples, tissue samples, or bodily fluids. The purification instrument operates in accordance with a purification plan associated with a run plan provided by the server. The purification instrument provides a transfer plate including nucleic acids disposed as solutions in wells of the transfer plate and a transfer file providing information about the nucleic acids disposed on the transfer plate. The sequencer receives the transfer plate, the transfer file, and a sequencing plan associated with the run plan. The sequencer sequences the nucleic acids in accordance with the sequencing plan based on the transfer file. The server can control the purification instrument and the sequencing instrument, monitor progress of the various operations, and perform post sequencing analysis, such as variant calling.

In an example, a user provides a run plan to the sequencing instrument. The run plan includes a purification plan and a sequencing plan. The purification instrument requests a set of run plans. A user can select a run plan from the set using a user interface of the purification instrument, and the purification instrument can perform its operations in accordance with the purification plan associated with the selected run plan. The purification instrument provides the purified nucleic acids on a transfer plate and provides to the server a transfer file identifying in which well of the plate nucleic acid solutions are located and optionally quantifying data associated with the nucleic acid solutions. The transfer plate can be moved to the sequencer. The sequencer can access the sequencing plan associate with the select run plan and can perform a sequencing operation in accordance with the selected sequencing plan and transfer file. Optionally, the sequencing plan includes a library preparation and sequencing operations. Upon completion of the sequencing operations, data derived from sequencing operations can be provided to the server to perform further sequence analysis.

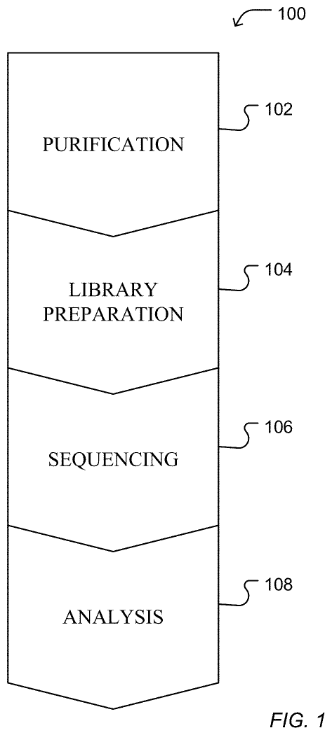

As illustrated in FIG. 1, a set of processing steps can be used to provide a sequencing analysis of nucleic acids extracted from various sources. In an example, a source can include an environmental source, such as water, soil, organic materials. In another example, a source can be a tissue or bodily fluid. For example, the tissue may be an FFPE sample. In another example, bodily fluid can include blood, mucus, urine, fecal matter, saliva, amniotic fluid, spinal fluid, or bone marrow, among others.

As illustrated at block 102 of FIG. 1, nucleic acids can first be isolated and purified from the source, using a purification instrument. The purification instrument can perform various operations to isolate nucleic acids from samples. For example, the purification instrument can use magnetic beads to capture nucleic acids and isolate them from other components of the tissue sample. Optionally, the purification instrument can quantify the amount of nucleic acids within a given solution. For example, the purification instrument can determine a concentration of nucleic acids within the extracted solution.

Optionally, as illustrated at block 104, purified nucleic acids can undergo library preparation. For example, primer compliments can be added at terminal ends of isolated nucleic acids. Particular nucleic acids can be amplified or target regions of the nucleic acids can be amplified.

The prepared library of nucleic acids can be sequenced, as illustrated at block 106. In an example, a sequencer can secure monoclonal populations of nucleic acids on plates or within wells. In some examples, the sequencer can operate by sequencing-by-synthesis. Example systems include optical systems or semiconductor-based systems. In an example, the sequencer implements measurement of sequencing-by-synthesis utilizing ion sensitive field effect transistor of monoclonal populations disposed within wells. An example of such system is described in US Published Application No. 2019/0255505A1, which is incorporated by reference in its entirety.

Following the measurement of signals derived from the sequencing-by-synthesis reactions, a server can further analyze the data, as illustrated at block 108, making base calls, identifying variant, and identifying aspects of the sequence.

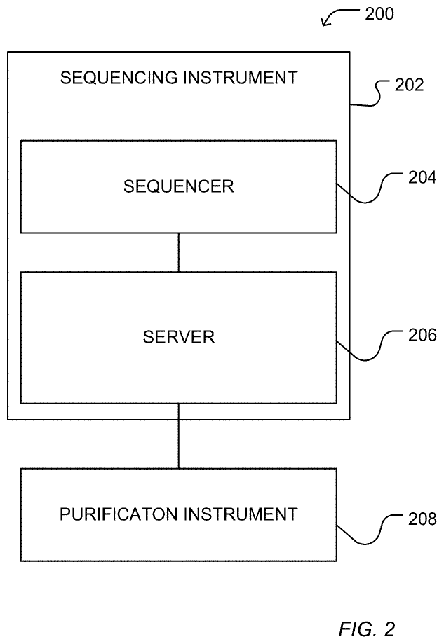

FIG. 2 includes an illustration of an example system 200 for determining nucleic acid sequences from samples. As illustrated, the example system 200 includes a sequencing instrument 202 that incorporates a sequencer 204 and a server 206. The system 200 further includes purification instruments 208. As illustrated, the purification instrument 208 is separate from the sequencer 204. Alternatively, the purification instrument 208 can be incorporated in the same housing as the sequencer 204. In a further alternative, the server 206 can be housed separately from the sequencer 204. For example, the server 206 may interact with the sequencer 204 and a purification instrument 208 via Internet or network connection. In particular embodiments, the server may reside on a cloud accessible to the purification instrument 208 and the sequencer 204.

In an example, a user establishes a run plan on the sequencer instrument 202. The run plan includes a purification plan and a sequencing plan. The run plan can be stored with a set of run plans. In operation, a user can direct the purification instrument 208 to request a set of run plans from the server 206. The server 206 provides the run plans to the purification instrument 208, and a user can select a run plan from the set of run plans. The purification instrument can then operate in accordance with the purification plan associated with the selected run plan. Optionally, the purification instrument automatically observes the available consumables to determine whether the appropriate consumables have been provided to the instrument to perform the purification plan associated with the select run plan. The purification instrument 208 isolates and purifies nucleic acids derived from the samples provided to the instrument. Optionally, the purification instrument 208 can quantify isolated nucleic acids. The isolated nucleic acids are provided as nucleic acid solutions disposed in wells of a transfer plate, and a transfer file is provided that includes information about the location and nature of a given nucleic acid solution and optionally a quantification of that nucleic acid solution.

The transfer plate is moved to the sequencer 204 and the transfer file is provided to the server 206. The sequencer 204 can access the sequencing plan associated with the run plan and can perform sequencing in accordance with the sequencing plan and the transfer file. Optionally, the sequencer 204 performs library preparation, followed by sequencing-by-synthesis. Sequencing information derived from sequencing-by-synthesis can be provided to the server that performs further analysis, for example, determining sequences and calling variance.

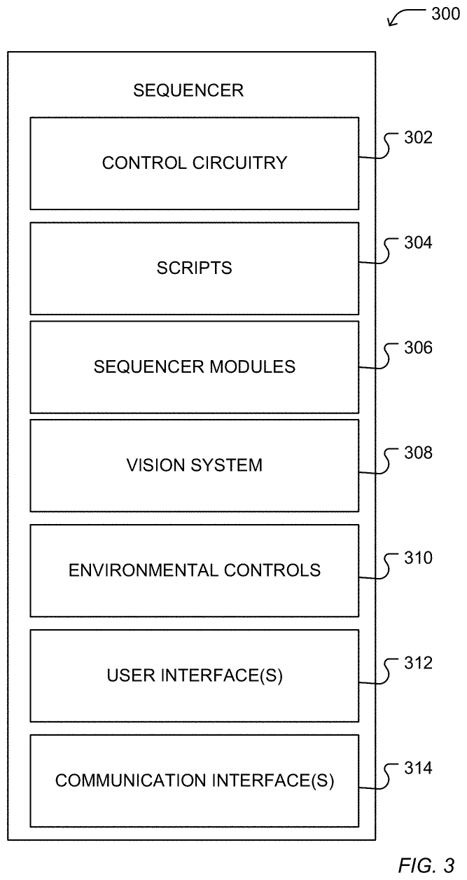

FIG. 3 includes an illustration of a sequencer 300. The sequencer 300 can include a control circuitry 302 that utilizes scripts 304 to control sequencing modules 306, a vision system 308, environmental system 310, user interfaces 312, and communication interfaces 314. In particular, the sequencer 300 can activate particular scripts 304 based on a sequencing plan of a run plan. The sequencer modules 306 can utilize various subsystems to prepare a library of the extracted and purified nucleic acids and utilize the library to perform sequencing of the nucleic acids or targeted regions thereof. In an example, the sequencing modules 306 can include multi-axis pipetting robots to perform library preparation and prepare nucleic acids for sequencing. The modules can also include thermal and fluidic systems to facilitate sequencing-by-synthesis.

Optionally, the sequencer 300 includes a vision system 308. In an example, the vision system 308 can observe consumables placed within the sequencer and can determine whether the appropriate consumables have been placed in the instrument to perform the sequencing plan. As such, the sequencer 300 can automatically determine whether the appropriate consumables have been placed in the instrument using the vision system 308.

The sequencer 300 may also include environmental controls 310. Such environmental controls 310 control the temperature within the instrument or can utilize airflow or UV lamps to clean the instrument.

The sequencer 300 further includes user interfaces 312. In an example, user interface can include a keyboard, a mouse, a touchpad, electronic pens, touchscreens, speaker, microphones, or displays, among other interface devices. In particular, the sequencer 300 can permit a user to enter a run plan on the sequencer 300 through a user interface 312. Such a run plan can be stored with a set of run plans by server.

Further, the sequencer 300 can include communications interfaces 314. In particular, the communications interfaces 314 can interact with a server to provide and retrieve run plans, provide status updates related to run plans, retrieve transfer files, and provide sequencing data. The communications interfaces 314 can include wired or wireless interfaces. For example, the communications interfaces 314 can include ethernet or universal serial bus interfaces. In another example, the communications interfaces 314 can include wireless interfaces, such as interfaces conforming to IEEE 802.11x.

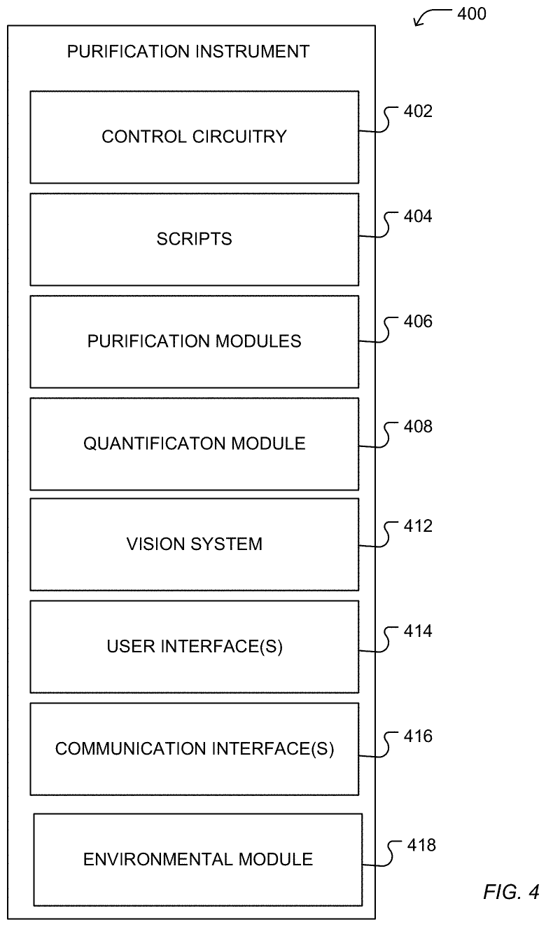

FIG. 4 illustrates a purification instrument 400 that includes a control circuitry 402 and scripts 404 to control purification modules 406, quantification module 408, vision system 412, user interfaces 414, communication interfaces 416, and environmental module 418. Scripts 404 can be used to implement a purification plan associated with select run plan received from a server through the communications interfaces 416. In an example, the purification module 406 includes a system that isolates and washes nucleic acids utilizing magnetic beads. The quantification module 408 can determine the presence of an amount of nucleic acids within the solution. The purification instrument can provide the purified nucleic acids in the form of nucleic acid solutions to a transfer plate and can prepare a transfer file that includes information about which solution is disposed in which wells of the transfer plate. Further, the transfer file can include quantification information about the nucleic acid solutions disposed within the wells of the transfer plate.

A vision system 412 can optionally be used to automatically determine whether the correct consumables have been installed to implement purification plan. For example, the vision system can recognize codes, such as barcodes or QR codes. Alternatively, the vision system can use shape and color determine the presence of the correct consumables.

The purification instrument 400 includes user interfaces 414. Such user interfaces 414 can include a keyboard, a mouse, a touchpad, electronic pens, touchscreens, speaker, microphones, or displays, among other interface devices.

The communication interfaces 416 of the purification instrument 400 can interact through a network with the server. In an example, the server provides run plans, receives progress updates from the purification instrument 400, and receives the transfer file, via the communications interfaces 416. The communications interfaces 416 can include wired or wireless interfaces. For example, the communications interfaces 416 can include ethernet or universal serial bus interfaces. In another example, the communications interfaces 416 can include wireless interfaces, such as interfaces conforming to IEEE 802.11x.

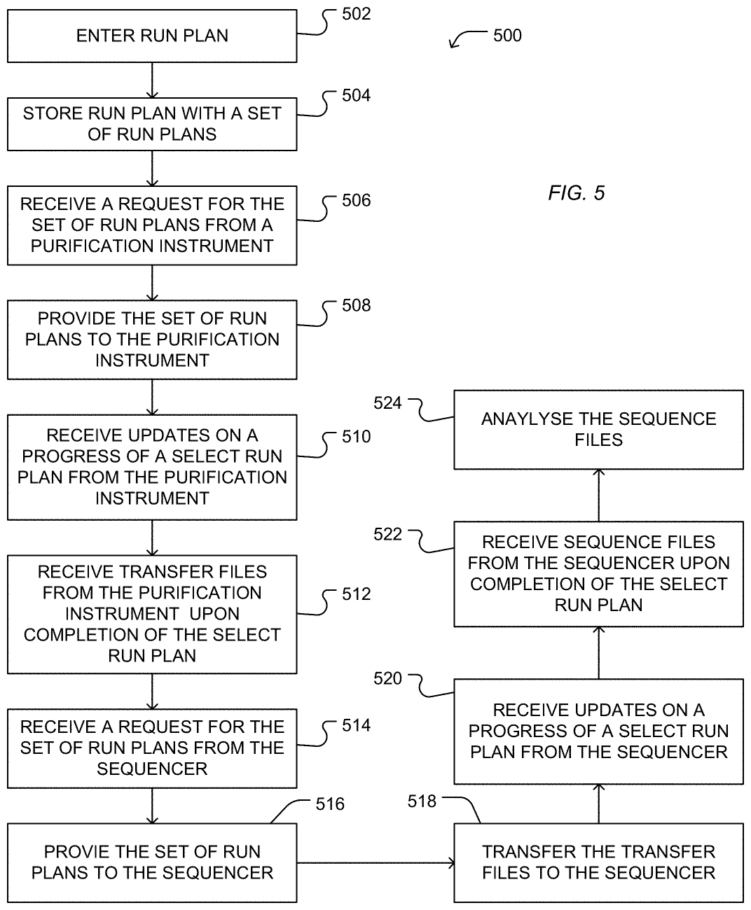

In an example, a method 500 illustrated in FIG. 5 is implemented by a server of a sequencing system. As illustrated at block 502, a user can enter a run plan. For example, the user can enter a run plan on the sequencer instrument. As illustrated at block 504, the run plan is stored with a set of run plans. The run plan can include a purification plan and a sequencing plan. Optionally, a sequencing plan can include a library preparation plan and a sequencer plan. Alternatively, the library preparation plan be separate from the sequencing plan.

As illustrated at block 506, the server can receive a request for the set of run plans from the purification instrument. The server can provide the set of run plans to the purification instrument, as illustrated at block 508.

The purification instrument can implement a purification plan associated with a select run plan, and the server can provide the select run plan or the purification plan of the select run plan to the purification instrument.

As illustrated at block 510, the server can receive updates on the progress of the select run plan from the purification instrument. When the purification plan is complete, the server can receive a transfer file from the purification instrument, as illustrated at block 512. In particular, the transfer file can include information about in which well a nucleic acid solution is disposed. A nucleic acid solution is disposed on a transfer plate that is provided to a sequencer. Optionally, the transfer file can include a quantification information associated with the nucleic acid solution, such as a concentration of nucleic acid, and amount of the nucleic acid solution provided in the well.

The server can receive a request for the set of run plans from the sequencer, as illustrated at block 514. The server can provide the set of run plans to the sequencer, as illustrated at block 516. A user may select a run plan and implement the sequencer plan associated with the run plan. Based on a select run plan, the server can transfer the transfer files to the sequencer, as illustrated at block 518. The sequencer can sequence the nucleic acids following the sequencing plan associated with the run plan and utilizing the transfer files associated with a transfer plate on which samples are stored. In an example, the transfer file can be used to inform the library preparation process. For example, concentrations of nucleic acids stored in the transfer file can be used to adjust the library preparation process.

The server can receive updates on the progress of the select run plan from the sequencer, as illustrated at block 520. Further, upon completion of the select sequencing plan, the server can receive sequencing files from the sequencer, as illustrated at block 522. Such files can be used to perform analysis of the sequencing information, as illustrated at block 524. In an example, the run plan may further specify the analysis to be performed by the server. For example, the system can perform base calls. In another example, system can perform variant calls or discover relevant regions within a nucleic acid.

As illustrated in FIG. 6, a method 600 for providing purified nucleic acid samples includes requesting a set of run plans from the server, as illustrated and block 602. The server can provide a set of run plans, which are received by the purification instrument, as illustrated at block 604. The run plans can be displayed on user interfaces associated with purification instrument, as illustrated at block 606. Through the user interfaces, the purification instrument can receive a selection of a run plan from the user, as illustrated at block 608.

The run plan can include an associated purification plan. To perform the purification plan, a purification instrument utilizes a particular set of consumables. For example, the consumables can include reagents useful in isolating nucleic acids from particular sources and optionally quantifying the nucleic acids recovered from the samples. The purification plan can include an identifier associated with the run, a number of sources, an identifier for each source, a type of source for each source (e.g., blood, FFPE . . . ), the type(s) of nucleic acid to be extracted for each source (e.g., DNA, RNA, or both), whether quantification is to be used, the types of assays that will use the extracted nucleic acids, a reference to an assay definition file (ADF), whether excess extracted nucleic acid is to be archived, or any combination thereof. In particular, the purification instrument can extract nucleic acids from more than one source to be used with more than one assay. As such, the purification plan can identify a first source (sample) and a first type of nucleic acid to be extracted and can identify a second source (sample) and a second type of nucleic acid to be extracted, wherein nucleic acid extracted from each of the sources (samples) is stored on the same transfer plate. In an example, the first and second types of nucleic acid are different. In a further example, the purification plan can direct the instrument to extract both DNA and RNA from the same sample.

Optionally, as illustrated at block 612, the purification instrument can perform a check for the presence of consumables associated with the purification plan. In an example, the purification instrument can include a vision system that recognizes consumables. For example, a vision system can recognize consumables by shape or optionally by codes, such as a barcode, QR code, or other identifiers. The consumables can include a transfer plate and an archive plate. In an example each of the transfer plate and archive plate have unique identifiers readable by the vision system of the purification instrument. An example of such a system can be found in US Patent Publication No. 2020/0075130A1, which is incorporated herein in its entirety. Alternatively, the purification instrument can include electric contacts or weight sensors to recognize the presence or absence of consumables to be used in conjunction with performing the purification plan.

As illustrated at block 614, the purification system can perform the purification plan of the selected run plan. For example, the purification system can extract nucleic acids, such as DNA or RNA, from the sources in accordance with the purification plan. Performing the purification plan includes providing the purified nucleic acid solutions to a transfer plate or wells of the transfer plate and optionally storing excess purified nucleic acid solutions in an archive plate.

The purification system can provide status updates to the server, as illustrated at block 616. For example, the purification system can periodically notify the server of the status of a particular purification plan. In another example, the purification system can update the server based on performance steps associated with the purification plan.

Optionally, the purification system can include a quantification unit. For example, a fluorescent detection system can be utilized to quantify an amount of nucleic acids within a particular nucleic acid solution.

The purification system 620 can save information to transfer files. In particular, the transfer files can include an identification of the transfer plate and a well location of nucleic acids solutions on the transfer plate and associated identifiers relating to the source (e.g., a number associated with a blood sample of an individual). Optionally, the transfer file can store a nucleic acid type, a concentration or amount of nucleic acids, or a methodology used to acquire the nucleic acid solution for each of the wells of the transfer plate. Further, the transfer file can include information relating to which run it is associated, nucleic acid sources, batch of consumables, transfer plate identifier, archive plate identifier, archive well locations of nucleic acids, concentrations, or purification instrument identifier, among other information.

As illustrated at block 620, the purification instrument can provide the transfer file to the server. For example, the purification instrument can notify the server of the existence of the transfer file and can transfer the transfer file using an FTP protocol.

As illustrated in FIG. 7, a method 700 for sequencing nucleic acids includes requesting a set of run plans from the server, as illustrated at block 702. The server can provide the run plans to the sequencer. For example, the sequencer can receive a set of run plans, as illustrated at block 704, and can display the set of run plans to the user, as illustrated at block 706. The user can select a run plan using user interfaces of the sequencer, as illustrated at block 708. The selected run plan can include an associated sequencing plan which optionally can include library preparation and sequencing directives.

In an example, the sequencing plan can include an identifier associated with the run plan, an identifier associated with each source, types of nucleic acid associated with each source (e.g., RNA, DNA or both), indication of an assay to be used for each source, nucleic acid tags or barcodes to be associated with each source, a type of sequencing chip, allocation of lanes when the sequencing chip has multiple lanes, a reference to one or more assay definition files, or any combination thereof. In another example, the system can automatically assign nucleic acid tags or barcodes to samples. In a particular example, the sequencing plan includes an identifier associated with each source and an assay to be used for each source. The system can then reference ADF files associated with the assays and seek the source nucleic acids in the transfer file.

The system can sequence nucleic acids (e.g., DNA) derived from more than one sample and more than one nucleic acid type (e.g., DNA or RNA) using more than one assay. As such, the sequencing plan may reference a first sample, a first type of nucleic acid, and a first assay, and may reference a second sample, a second type of nucleic acid, and a second assay, wherein the first type of nucleic acid is different from the second type of nucleic acid or the first assay is different from the second assay.

When the nucleic acid solutions provided to the sequencer are derived from the purification instrument, a transfer plate from the purification instrument is provided to the sequencer. The sequencer can retrieve the associated transfer files from the server, as illustrated at block 710. For example, the sequencer can access an FTP server of the server and acquire an associated transfer file.

To perform a sequencing plan, the sequencer utilizes a particular set of consumables. Optionally, the sequencer can perform a check for the appropriate consumables, as illustrated at block 712. For example, the sequencer can include a vision system that recognizes the consumables and their appropriate positioning within the sequencer. For example, sequencer can visually observe shape of the consumables. In another example, the sequencer can read codes on the consumables, such as barcodes or QR codes. In a further example, consumables may be tagged with RFID codes that can be read by the sequencer. An example of such a system can be found in US Patent Publication No. 2020/0075130A1, which is incorporated herein in its entirety.

As illustrated at block 714, the sequencer can perform a sequencing plan of the selected run plan based on the information of the transfer file. For example, the sequencer can dilute portions of the nucleic acid solutions based on quantification identified in the transfer file. Further, the sequencer can modify performance of library preparation and sequencing functions based on the types of nucleic acids, the methods for extracting such nucleic acids, or the source of the nucleic acids.

As the sequencer performs the sequencing plan, the sequencer can provide status updates to the server, as illustrated at block 718. For example, the sequencer can provide periodic updates to the server. In another example, the sequencer can provide updates to the server based on progress along the sequencing plan.

Upon completion, the sequencer can store sequencing data within sequencing files, as illustrated at block 718. The sequence files can be provided to the server, as illustrated at block 720. For example, the sequence files can be transferred using enough to include process to the server. The server can utilize sequence files to perform sequence analysis. For example, the server can determine variant calls.

In an example embodiment, a purification instrument includes a pipetting system having three axis movement, a sled mechanism configured to select comb magnets from a pair of comb magnets, a deck including supports for securing protective comb covers, receptacles to receive a first type of welled plates, and receptacles to receive a second type of welled plates. The instrument can further include a fluorometer and an associated receptacle to store reagents. In addition, the deck of the instrument can include a receptacle to receive a transfer plate and a receptacle to receive an archive plate. The deck may also include receptacles to receive trays of pipette tips.

The purification instrument can extract nucleic acids, such as deoxyribonucleic acid (DNA) or ribonucleic acid (RNA), by selecting a magnetic comb using a slide mechanism. The instrument can select a magnetic comb based on a type of samples to be extracted. Using the pipette system, samples and reagents can be mixed. Reagents can include magnetic particles. Utilizing the selected magnetic comb, nucleic acids coupled to magnetic particles can be separated from other components within the sample. A concentration of the extracted nucleic acids can be determined utilizing the quantification fluorometer. A portion of the extracted samples can be stored on the transfer plate to be transferred to a sequencing instrument. Remaining extracted solution can be stored on an archive plate.

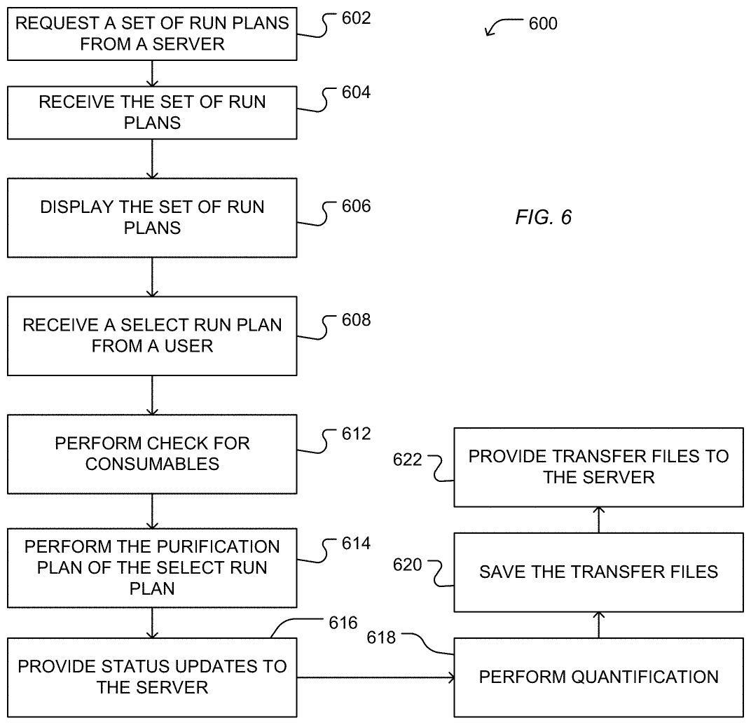

FIG. 8 includes an illustration of an example purification instrument 800 for extracting nucleic acids from samples. The purification instrument 800 includes an outer shell 802, a door 804 to access the inner workings of the purification instrument 804, and a user interface 806, such as a touchscreen interface. Alternatively, the user interface can include monitors, physical keyboards, or pointer devices. An example purification instrument 800 is described in U.S. application Ser. No. 17/526,979, which is incorporated herein in its entirety.

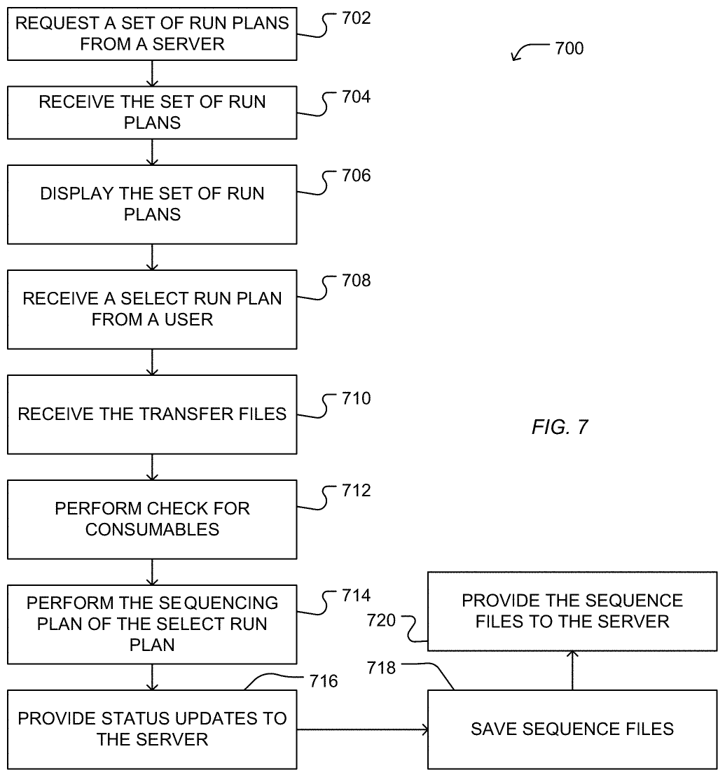

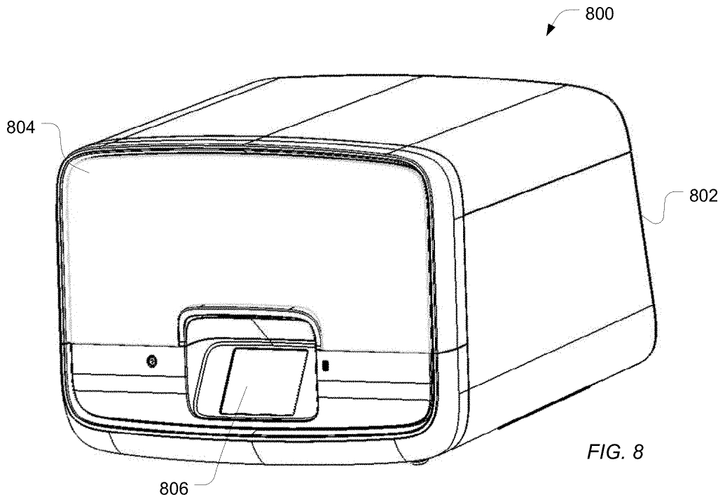

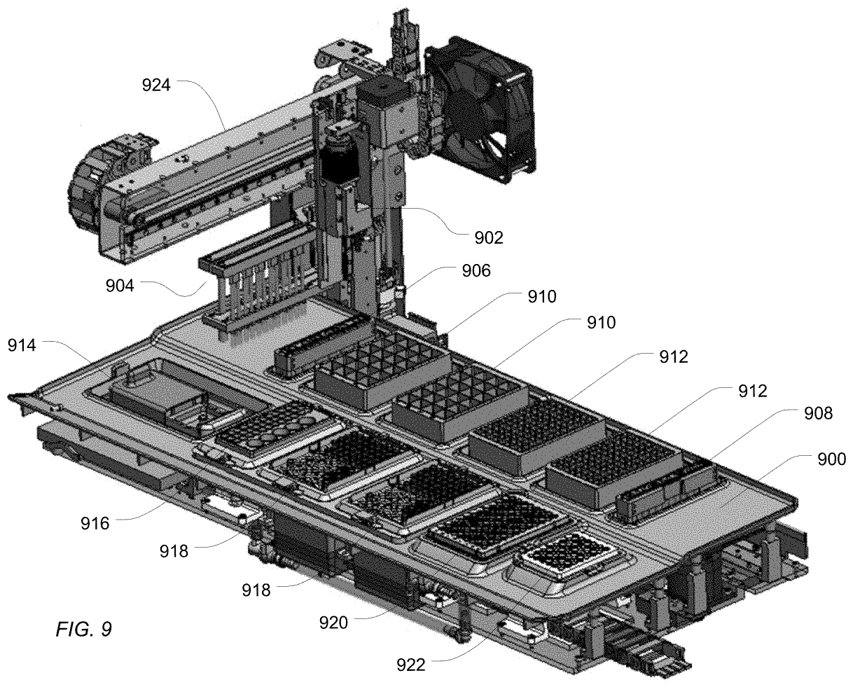

FIG. 9 includes an illustration of example inner workings of the purification instrument 800. For example, the system can include a deck 900 and a pipetting system 902, including a three-axis gantry 924. In addition, the system can include a sled mechanism 904 for selecting a magnetic comb from a set of magnetic combs. The deck 900 can include receptacles 906 and 908 to secure magnetic comb covers. The deck 900 can also include receptacles for a set of first welled plates 910 and second set of welled plates 912. For example, the first welled plates 910 can be 24-well plates. In another example, the second welled plates 912 can be 96-well plates. The deck 900 can also include receptacles for arrays 918 of pipette tips.

Optionally, the system includes a fluorometer 914. The deck 900 can include a receptacle for a reagent plate 916 for storing reagents for use with the fluorometer 914. Extracted nucleic acid samples can be stored on a transfer plate 920. Remaining extracted solutions can be archived on the archive plate 922.

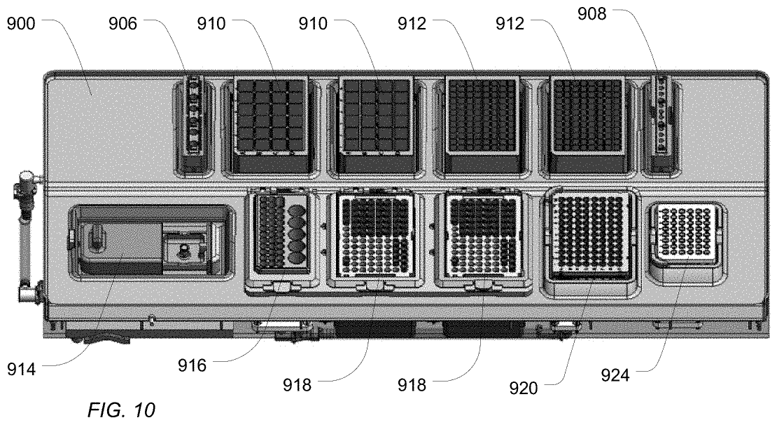

As illustrated in FIG. 10, the deck 900 includes cover supports 906 and 908 to hold the protective covers for the magnetic combs. In addition, the system can security different types of plates 910 or 912, including a different number of wells. For example, the plate 910 can include an array of 24 wells, while the plate 912 can include 96 wells.

The deck 900 can further secure a quantitative fluorometer 914 and associated reagent plate 916 with wells for mixing reagents and extracted samples. Portions of the extracted nucleic acids can be stored in a transfer plate 920 and remaining portions of the extracted samples can be stored in an archive plate 922.

The receptacles for the various types of plates can include blocks for heating or cooling at least a portion of the wells of the plates. For example, receptacle to receive a plate 910 can include a temperature control block for heating or cooling rows of wells of the plate 912. The temperature control block can control the temperature at least two rows of wells. Similarly, receptacles to receive second type of plate 912 can include temperature control blocks to control the temperature of a number of rows, such as two rows, of the wells of the plate 912. A receptacle to receive the reagent tray 916 can include one or more temperature control blocks to control the temperature of some or all of the wells of the reagent tray 916. Similarly, a receptacle can include temperature control block to control the temperature of one or more rows of the transfer plate 920, and a receptacle can include a temperature control block to control the temperature of rows of an archive plate 922.

The system can include a fluorometer 914. In particular, the fluorometer 914 can assist with quantifying a concentration of extracted nucleic acid in a given solution derived from a particular sample. In an example, a proportion of an extracted sample mixed with quantification reagents is inserted into a sample port. An automated lid may close while the fluorometer 914 takes a measurement.

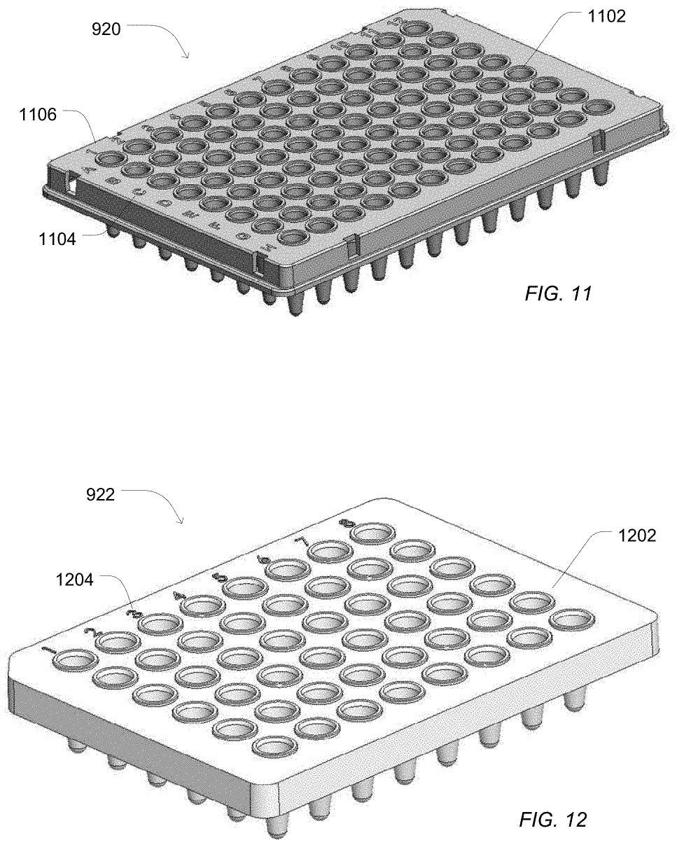

The system can also include a transfer plate of wells for storing extracted samples and an archive of wells for storing extracted samples for archiving. For example, as illustrated in FIG. 11, a transfer tray 920 for storing extracted samples for use by a sequencer can include an array of wells 1102. In addition, the tray 920 can include indicia 1104 of the rows and indicia 1106 of the columns. As illustrated in FIG. 12, an archive tray 922 can include an array of wells 1202 for storing extracted solutions for archiving. The tray 922 can include indicia 1204 indicative of columns and optionally rows. Generally, the archive tray of archive samples is frozen for later use.

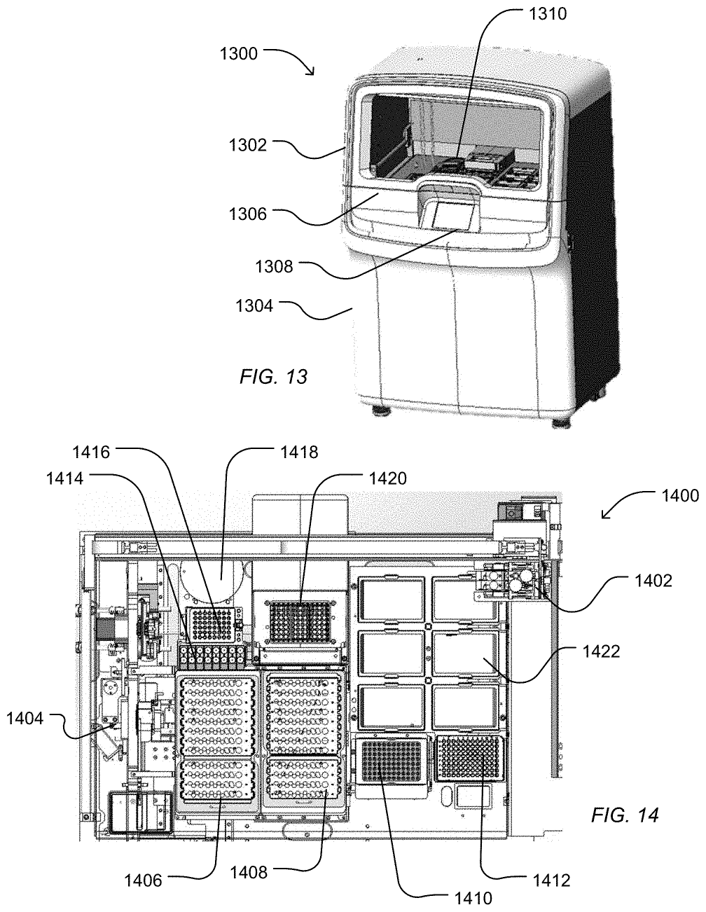

FIG. 13 includes an illustration of an example sequencing instrument 1300 incorporating a three-axis pipetting robot. In an example, the instrument 1300 can be a sequencer incorporating a sample preparation platform. For example, the instrument 1300 can include an upper portion 1302 and a lower portion 1304. The upper portion can include a door 1306 to access a deck 1310 on which samples, reagent containers, and other consumables are placed. The lower portion can include a cabinet for storing additional reagent solutions and other parts of the instrument 1300. In addition, the instrument can include a user interface, such as a touchscreen display 1308. An example sequencing instrument is described in US Patent Publication 2021/0054450A1, which is incorporated herein by reference in its entirety.

In a particular example, the sequencing instrument 1300 includes a top section, a display screen, and a bottom section. In some embodiments, the top section may include a deck supporting components of the sequencing instrument and consumables, including a sample preparation section, a sequencing chip and reagent strip tubes and carriers. In some embodiments, the bottom section may house reagent bottles used for sequencing and a waste container.

In some embodiments, one or more cameras mounted in a cabinet of the top section of the instrument is oriented towards the deck to monitor what items are in place in preparation for a sequencing run. The camera can acquire video or images at time intervals. For example, images may be acquired at 1-4 second intervals or any suitable interval. In another example, frames of a video stream can be extracted at intervals such as in a range of 0.5 seconds to 4 seconds. A computer or processor analyses images to detect the completion of a task by the user. The computer or processor may provide feedback and instructions for the next task in the preparation via the display screen. The display screen may present graphical representations of the instrument components and consumables in order to illustrate instructions for the user.

An example instrument deck 1310 is illustrated in FIG. 14 as instrument deck 1400. The deck 1400 is housed in the top section of the instrument in the view of the camera or cameras. The sample preparation deck may include a plurality of locations configured to receive reagent strips, supplies, a sequencing chip, and other consumables. As used herein, consumables are components used by the instrument that are replaced periodically as they are used. For example, consumables include reagent and solution strips or containers, pipette tips, microwell arrays, and flow cells and associated sensors, among other disposable components not part of the permanent components of the instrument.

In an example, the deck 1400 includes a pipetting robot 1402 that accesses various reagent strips and containers, pipette tips, microwell arrays, and other consumables to implement a library preparation or sequencing run. Further, the deck can include mechanisms 1404 for carrying out testing. Example mechanisms 1404 include mechanical conveyors or slides and fluidic systems.

In an example, the deck 1400 includes trays 1406 or 1408 to receive solution or reagent strips of a particular configuration. In an example of a sequencing instrument, the tray 1406 can be used for library and template solutions in appropriately configured strips, and the tray 1408 can receive library and template reagents in the appropriate configuration.

Further, the instrument can be configured to receive microwell arrays 1410 and 1412 at particular locations on the deck. For example, a sample can be supplied in an array of wells, such as microwell array 1412, e.g., a transfer plate. In another example, the system can be configured to receive additional reagents 1414 in a different strip configuration. In another example, reagent solutions can be provided in an array 1416. In a further example, container arrays 1420 can be provided in conjunction with instrumentation, such as a thermocycler. Further, the system can include other instrumentation, such as a centrifuge, that may be supplied with consumables, such as tubes. Further, trays can be provided to receive pipetting tips 1422.

The appropriate provisioning of consumables in each of these locations can be monitored by a vision system including one or more cameras. The deck may be provided with one or more cameras to track provisioning and securing of reagents and other consumables. The user can be prompted through the user interface when a reagent is missing that is to be utilized to perform one plan or when a reagent consumable is present in a used state.

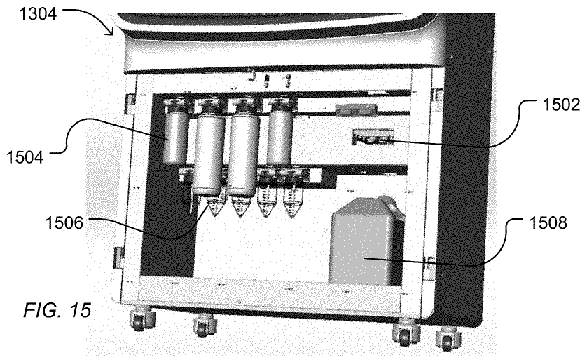

FIG. 15 includes an illustration of a reagent storage cabinet 1304 to store larger volume reagent and solution containers. For example, the cabinet storage 1304 includes an interface 1502 to receive a reagent cartridge. In another example, the storage 1304 can provide space for containers 1504 or 1506. In a further example, the storage 1304 can include space for a waste container 1508.

In some embodiments, a nucleic acid sequencing instrument may be interfaced with a server system for control of various components of the sequencing instrument and processing of data output from sequencing runs on the sequencing instrument. The server system software may include a web application, databases and analysis pipeline and support connections from a sequencing instrument (e.g., FIG. 13). The server system software may provide the following major functionalities and application program interfaces (APIs):

1. APIs for user authentication, reagent tracking, run information and run tracking/logging. Supported instruments may include the sequencing instrument and extraction or purification instrument.

2. APIs for a LIMS (Laboratory Information Management System) for creation of samples, libraries, plan run and retrieve the run status of the plan.

3. Support for management of samples and run data.

4. Support for assay configuration and execution of the analysis pipeline for data analysis and reporting.

5. Interface to a software update server for software updates and maintenance.

6. Supports configuration to connect to an annotation and reporting system, such as Ion Reporter from Thermo Fisher Scientific, deployed in a cloud-based system or a local system, and establishes secure and authenticated connection with the cloud-based system to transfer mapped or unmapped BAM files.

7. Supports configuration to connect to a resource system in a cloud computing environment, such as the Thermo Fisher Cloud, and establishes secure and authenticated connection with the cloud resource system to download software and system contents and to send telemetry data.

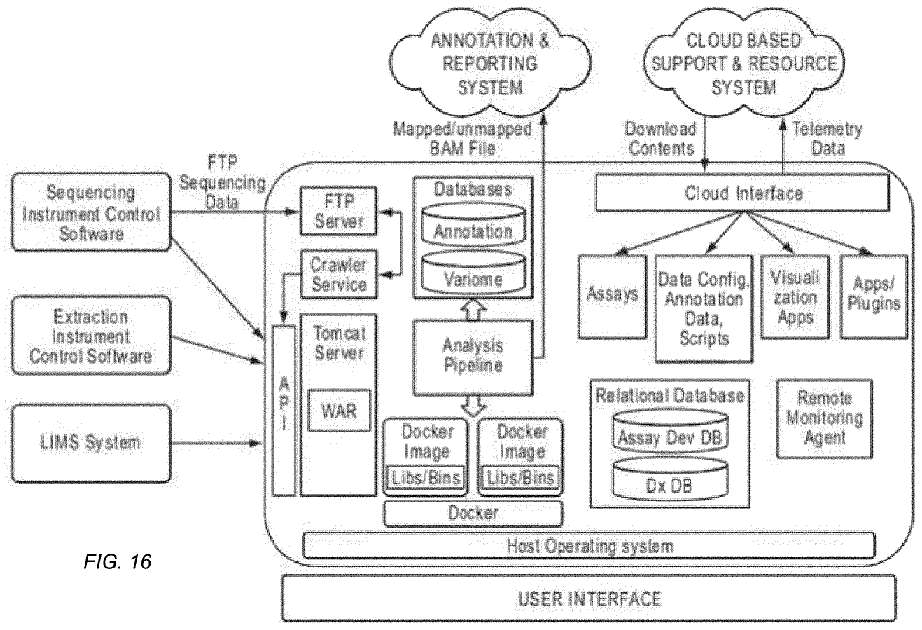

FIG. 16 includes a schematic diagram of the server system components. In some embodiments, the basic software architecture may comprise a web interface, remote monitoring agent, databases, APIs to the instruments, analysis pipeline, containerization of the analysis pipeline (using Docker, for example), connectivity to an annotations and reporting system (e.g. Ion Reporter from Thermo Fisher Scientific) and a cloud-based support and resource system (e.g. Thermo Fisher Cloud). The cloud-based support and resource system, or cloud-based resource system, may be implemented in a cloud computing and storage system. The cloud-based support and resource system stores content including assay definition files. A server of the cloud computing and storage system may download contents, such as assay definition files, to the local server system. The cloud-based support and resource system may receive telemetry data from the local server system. Server system, local server system and user's server system are used interchangeably herein.

In some embodiments, a user interface (UI) may be implemented via web application software. The UI may provide sample management pages. The sample management UI pages allow the user to enter sample information into the system. Sample information includes unique sample identifier (ID), sample name and sample preparation reagent tracking information. Validation logic is built into the sample management flow that locks the sample preparation step to the pre-defined assay workflow. The UI may provide assay management pages. Assay management UI pages allow the user to view assays, and create assays. The assays lock the workflows to pre-defined parameters for each step of the process. Validation logic may be built in to ensure the assay configuration. The UI may provide run plan and monitor pages. The run plan and monitor UI pages allow the user to plan for a run and monitor the run in progress. The UI may provide output data pages. The output data UI pages allow the user to view the analysis results along with quality control (QC) metric evaluation, log and audit trail of the results generated. The UI may provide configuration pages. The configuration UI pages allow users to view and configure the system.

In some embodiments, application programming interfaces (APIs) may be provided through a Java platform. For example, the Java platform may include a Tomcat server that may be used to build a Web ARchive (WAR) file for web-based applications.

Code modules for various steps of the analysis pipeline may be referred to as actors in the context of a Kepler workflow engine. For example, a code module for an analysis step may implemented by Java program binary code included in an actor jar. A Kepler workflow engine defines processing components of a workflow as “actors” and chains the steps for execution by a processor of the algorithm or analysis pipeline (kepler-project.org). For example, a Kepler workflow engine may be used to configure the workflow of the analysis pipeline in FIG. 16.

The server system may include one or more databases. For example, the server system may include a relational database for storing sample data, run data and system/user configuration. The relational database may include two separate databases: an assay development database and a Dx database. The assay development database may store sample data, run data and system/user configuration for RUO, or assay development, mode of operation. The Dx database may store sample data, run data and system/user configuration for the IVD, or Dx, mode of operation.

The server system may include an annotations database, AnnotationDB, for storing annotation source data. For example, the annotations database may be implemented as NoSQL, or non-relational, database, e.g. a MongoDB database. Each annotation source may be stored as a JSON (JavaScript Object Notation) string with meta information indicating source name and version. Each annotation source may contain a list of annotations keyed to annotation IDs. The server system may include a variome database, VariomeDB, for storing variant information. For example, the variome database may be implemented as a NoSQL, or non-relational, database, e.g. a MongoDB database. The VariomeDB may store a collection of variant call results on a particular sample. For example, a JSON formatted record may contain meta information for identifying the sample.

For example, the AnnotationDB database may store one or more of the following annotation sources:

1. RefGene Model: hg19_refgene_63, version 63

2. RefGene Functional Canonical Transcripts Scores: hg19_refgeneScores_4, version 4

3. dbSNP: dbsnp_138, version 138

4. Canonical RefSeq Transcripts: hg19_refgene_63, version 63

5. 5000Exomes: hg_esp6500_1, version 1

6. ClinVar: clinvar_1, version 1

7. DGV: dgv_20130723, version 20130723

8. OMIM: omim_03022014, version 03022014

Other annotation sources may be included. Other versions of the above annotation sources may be included. The annotation source may provide public annotation information content or proprietary annotation information content.

For each call in Variome database, and each annotation source may be queried for annotations matching the variant and matching annotations may be stored as key-value pairs in Variome database with the variant. Annotated variants may be included in a results file, e.g. an annotated VCF file, for the user. VCF files are tab-separated text files used for storing gene sequence variants. In some embodiments, the annotation methods for use with the present teachings may include one or more features described in U.S. Pat. Appl. Publ. No. 2016/0026753, published Jan. 28, 2016, incorporated by reference herein in its entirety.

In some embodiments, the server system may include an analysis pipeline to process sequencing data generated during a sequencing run for an assay performed by a sequencing instrument. The sequencer transfers sequencing data files and experiment log files to the server system memory, for example in raw .dat files, already processed .dat files producing block wise 1.wells files, and thumbnail data. The analysis pipeline accesses the data files from memory and starts data analysis for the run.

In some embodiments, a Docker container and Docker images may be used for packaging the analysis pipeline and operating system specific binaries. The Docker is a tool used to create, deploy, and run applications by using containers. Containers enable an application with all the parts it needs, such as libraries and other dependencies, to be bundled as one package. This allows applications software to use the same Linux kernel as the host system. The Docker image files may be packaged with libraries and binaries needed by the analysis pipeline code. The Docker may be used to adapt an application or algorithm to a new or different version of an operating system (OS) to create a Docker image of the application that is compatible with the OS version.

In some embodiments, the server system may include a crawler service for data transfer from the sequencing instrument to the analysis pipeline. The crawler is an event-based service that may be developed using JAVA NIO watcher API (application programming interface). NIO (Non-blocking I/O) is a collection of Java programming language APIs that offer features for intensive input/output (I/O) operations. The crawler may monitor the FTP directory configured for the sequencing instrument to transfer run data from the sequencing instrument to the analysis pipeline.

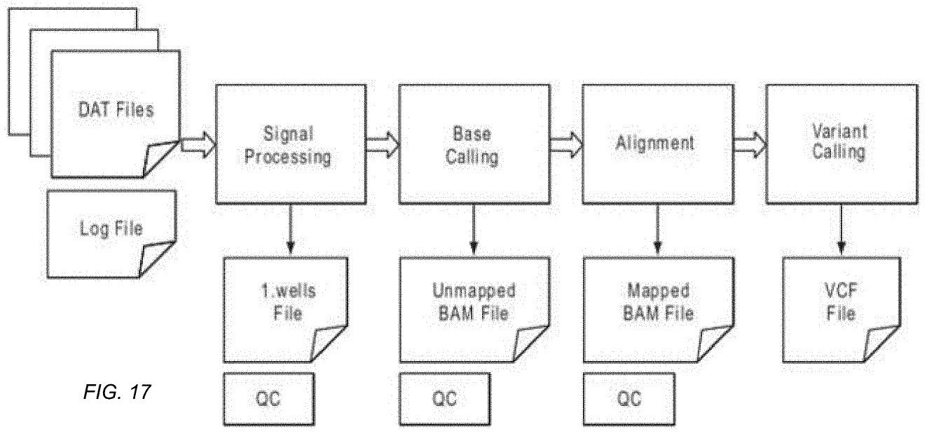

FIG. 17 is a block diagram of the analysis pipeline, in accordance with an embodiment. The sequencing instrument generates raw data files (DAT, or .dat, files) during a sequencing run for an assay. Signal processing may be applied to raw data to generate incorporation signal measurement data for files, such as the 1.wells files, which are transferred to the server FTP location along with the log information of the run. The signal processing step may derive background signals corresponding to wells. The background signals may be subtracted from the measured signals for the corresponding wells. The remaining signals may be fit by an incorporation signal model to estimate the incorporation at each nucleotide flow for each well. The output from the above signal processing is a signal measurement per well and per flow, that may be stored in a file, such as a 1.wells file.

In some embodiments, the base calling step may perform phase estimations, normalization, and runs a solver algorithm to identify best partial sequence fit and make base calls. The base sequences for the sequence reads are stored in unmapped BAM files. The base calling step may generate total number of reads, total number of bases and average read length as QC measures to indicate the base call quality. The base calls may be made by analyzing any suitable signal characteristics (e.g., signal amplitude or intensity). The signal processing and base calling for use with the present teachings may include one or more features described in U.S. Pat. Appl. Publ. No. 2013/0090860 published Apr. 11, 2013, U.S. Pat. Appl. Publ. No. 2014/0051584 published Feb. 20, 2014, and U.S. Pat. Appl. Publ. No. 2012/0109598 published May 3, 2012, each incorporated by reference herein in its entirety.

Once the base sequence for the sequence read is determined, the sequence reads may be provided to the alignment step, for example, in an unmapped BAM file. The alignment step maps the sequence reads to a reference genome to determine aligned sequence reads and associated mapping quality parameters. The alignment step may generate a percent of mappable reads as QC measure to indicate alignment quality. The alignment results may be stored in a mapped BAM file. Methods for aligning sequence reads for use with the present teachings may include one or more features described in U.S. Pat. Appl. Publ. No. 2012/0197623, published Aug. 2, 2012, incorporated by reference herein in its entirety.

The BAM file format structure is described in “Sequence Alignment/Map Format Specification,” Sep. 12, 2014 (github.com/samtools/hts-specs). As described herein, a “BAM file” refers to a file compatible with the BAM format. As described herein, an “unmapped” BAM file refers to a BAM file that does not contain aligned sequence read information and mapping quality parameters and a “mapped” BAM file refers to a BAM file that contains aligned sequence read information and mapping quality parameters.

In some embodiments the variant calling step may include detecting single-nucleotide polymorphisms (SNPs), insertions and deletions (InDels), multi-nucleotide polymorphisms (MNPs) and complex block substitution events. In various embodiments, a variant caller can be configured to communicate variants called for a sample genome as a *.vcf, *.gff, or *.hdf data file. The called variant information can be communicated using any file format as long as the called variant information can be parsed or extracted for analysis. The variant detection methods for use with the present teachings may include one or more features described in U.S. Pat. Appl. Publ. No. 2013/0345066, published Dec. 26, 2013, U.S. Pat. Appl. Publ. No. 2014/0296080, published Oct. 2, 2014, and U.S. Pat. Appl. Publ. No. 2014/0052381, published Feb. 20, 2014, and U.S. Pat. No. 9,953,130 issued Apr. 24, 2018, each of which is incorporated by reference herein in its entirety. In some embodiments, the variant calling step may be applied to molecular tagged nucleic acid sequence data. Variant detection methods for molecular tagged nucleic acid sequence data may include one or more features described in U.S. Pat. Appl. Publ. No. 2018/0336316, published Nov. 22, 2018, incorporated by reference herein in its entirety.

In some embodiments, the analysis pipeline may include a fusion analysis pipeline for fusion detection. Fusion detection methods may include one or more features described in U.S. Pat. Appl. Publ. No. 2016/0019340, published Jan. 21, 2016, incorporated by reference herein in its entirety. In some embodiments, the fusion analysis pipeline may be applied to molecular tagged nucleic acid sequence data. Fusion detection methods for molecular tagged nucleic acid sequence data may include one or more features described in U.S. Pat. Appl. Publ. No. 2019/0087539, published Mar. 21, 2019, incorporated by reference herein in its entirety.

In some embodiments, the analysis pipeline may include a copy number variants analysis pipeline for detection of copy number variations. Methods for detection of copy number variation may include one or more features described in U.S. Pat. Appl. Publ. No. 2014/0256571, published Sep. 11, 2014, U.S. Pat. Appl. Publ. No. 2012/0046877, published Feb. 23, 2012, and U.S. Pat. Appl. Publ. No. US2016/0103957, published Apr. 14, 2016, each of which is incorporated by reference herein in its entirety.

In some embodiments, the server system software may support an encapsulated assay configuration that includes assay name, assay type, panel, hotspot file if any, reference name, control names if any, quality control QC thresholds, assay description if any, data analysis parameters and values, instrument run script names and other configurations that define the assay. The entire set of the information is called an assay definition. The assay configuration content and corresponding workflows may be delivered to the user as modular software components in an assay definition file (ADF). The server system software may import an assay definition file that contains the assay configuration. The import process may be initiated by zip file import which includes an encrypted Debian file and triggers an installation process. The user interface may provide a page for the user to select an ADF for import. An application store in the cloud-based support and resource system may store ADFs supporting various assays, panels, and workflows available for selection by the user for download to the user's local server system.

An assay definition file (ADF) is an encapsulated file that defines configurations for the molecular test or assay, including assay name, technology platform configuration (for example, next generation sequencing (NGS), chip type, chemistry type), workflow steps (sample prep, instrument scripts, analytics, reporting), analysis algorithms, regulatory labels (for example, research use only (RUO), in vitro diagnostics (IVD), Central Europe in vitro diagnostics (CE-IVD, internal use only (IUO), etc.), targeted markers (panel), reference genome version, consumables, controls, QC thresholds, reporting genes and variants. The ADFs provide a modular approach to building assay capabilities for the local sequencing instrument. The assay software may be provided by the ADF separately from the platform software of the sequencing instrument.

The advantages of using the ADF for assay configuration include the following:

Encapsulation of the assay workflow and analysis

Single click for installation

No revalidation required after software update for assay configuration because of the modular structure of the software by the Docker implementation allowing separation from the platform software

Multi-tiered encryption for secure delivery

Streamlined support of assay configurations for original equipment manufacturers (OEM)

Streamlined customization of reporting

Support of regional regulatory requirements

Plug-n-play format supports technology agnostic workflows

Enables rapid expansion of molecular test menu and assay adoption by laboratories

In some embodiments, the assay definition file (ADF) may include software code modules for one or more of the following steps 1) library preparation; 2) templating; 3) sequencing; 4) analysis; 5) variant interpretation; and 6) report generation. For the workflow steps of library preparation and templating, the ADF may include scripts for preparing libraries, templating, and enrichment of templated beads. For the workflow steps of sequencing and analysis the ADF may include Docker image packages of algorithm binary code and parameters for the analysis pipeline described with respect to FIG. 17. For the workflow step of variant interpretation, the ADF may include a list of annotation sources that may be used for analyzing and annotating variants. For the workflow step of report generation, the ADF may include report templates and image files for use when a generating a report.

The ADF may include for the instrument scripts for control of workflow steps on the sequencing instrument. For example, scripts may include parameters controlling the amount of pipetting and robotic control. The instrument scripts may be customized for the particular assay.

For example, for the sequencing and analysis steps, the ADF may include a Docker image of the end to end analysis pipeline. The Docker image may include OS specific libraries and binaries for the algorithms each step of analysis pipeline. The algorithm binaries may include steps of the analysis pipeline including signal processing, base calling, alignment, and variant calling, such as those described with respect to FIG. 17. In another example, the ADF Debian file may package certain code modules for a particular assay, such as code modules for signal processing, base calling and RNACounts.

The ADF may include scripts for configuration of reagent kits. These scripts support calculation of the consumables needed for a sequencing run. The configurations scripts included in the ADF may include one or more of the following:

Barcode set and chip

Library kit and consumables, including capability to associate sample control configuration, (e.g. sample inline control) and its QC parameters

Templating kit and consumables, including capability to associate internal controls and QC parameters

Sequencing kit, including capability to associate internal controls and QC parameters

The ADF may include one or more reference genome files. Examples of reference genomes include hg19 and GRCH38. The reference genome file may be packaged in the main ADF with the workflow information. Alternatively, the reference genome file may be packaged in a separate ADF that is supplementary to the main ADF.

The ADF may include code modules for workflows of fusion panels and fusion target region panels. The ADF may include fusion target region reference files and hotspot files for analysis.

The ADF may include assay parameters at various points of the workflow that may be configured by the user. The configurable parameters may be displayed in the user interface for adjustment by the user. New parameters may be added at any actor level. The configurable parameters may be passed to the analysis pipeline. Input formats for the configurable assay parameters may include one or more single string text, Boolean, multiline text, floating point, radio buttons, drop downs, and file uploads. For example, the file uploads may use file formats such as properties and .json.

The ADF may include QC parameters used for quality control and assay performance thresholds at various points in the workflow. For example, types of QC parameters include run QC parameters, sample QC parameters, internal control QC parameters and assay specific QC parameters. A QC parameter may be defined by one or more of a data type (e.g. integer, floating point), lower bound, upper bound and default value.

The ADF may include specified data tab columns for results presentation that are selected from the database for a given assay. The selected data tab columns support configuration of the user interface display of results and the columns to be included in the PDF reports for the assay. The ADF may include image files for results presentation for a given assay. The ADF may include support for multiple languages for the PDF reports. The ADF may include a download file list for any files to be generated by the analysis pipeline for a given assay. The file list for the sample or run may be displayed at the user interface. The ADF may include a gene list. The gene list may be used to display the known list of genes for a given cancer type at the user interface and in a PDF report.

The ADF may include a set of plugins to be used for a given assay. The ADF may specify a set of plugins and their versions. If the ADF does not specify a version of a plugin, the latest version of the plugin installed on the server system may be used for the given assay.

The ADF may include a new workflow template to support custom assay creation. The new workflow template may include a set of assay chevron steps. Parameters for the steps may be displayed.

The ADF may include a list of annotation sources and sets to support the configuration of new annotation sets. The ADF may include filter chains to be applied to variants detected by the analysis pipeline of a given assay. The ADF may include rulesets for annotation of variants.

The ADFs can be configured to support a number of different types of assays. Examples include, but are not limited to, oncology related assays (e.g., Oncomine assays from Thermo Fisher Scientific), immuno-oncology related assays (e.g., T-cell receptor (TCR), microsatellite instability (MSI) and tumor mutation load (TML)), infectious diseases related assays (e.g. microbiome), reproductive health related assays and exome related assays. The ADF can also be configured for a custom assay.

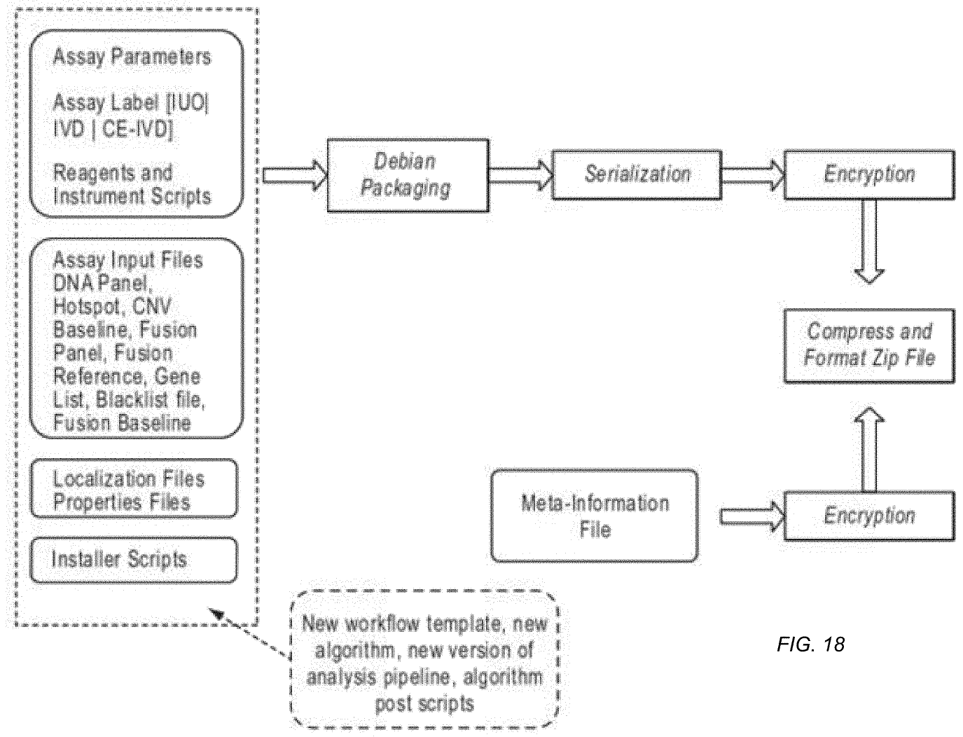

FIG. 18 is a schematic diagram of generating an assay definition file, in accordance with an embodiment. The assay definition may be generated by build.sh, debscripts and makedeb.sh that initiate file copying and database population of assay information to form a Debian file. The assay definition content may include assay parameters, BED files (Browser Extensible Data file—BED file—defines chromosome positions or regions), panel files, gene lists, hotspot files (a BED or a VCF file that defines regions in the gene that typically contain variants), and seed data containing allowable reagents. The assay definition content may contain localized versions of an assay name, description and report messages that support assay information display in different languages. The assay definition file may support the packaging of a new analysis pipeline. The ADF may include an optional post processing script which may be executed for variant calling, fusion calling and CNV calling based on the type of assay. The ADF may include an optional Docker container image of updates to the binaries for a specific analysis pipeline. The Docker container image may be packaged with the ADF to ensure that platform changes such as operating system or third-party library do not impact the results of the assays or functioning of the system.

The Debian file may be serialized to prevent unauthorized modifications. The serialized assay definition may be further encrypted using Advanced Encryption Standard (AES), a symmetric-key algorithm. A text file containing assay meta-information may also be encrypted using AES and the same encryption key. The encrypted assay definition file, together with the encrypted meta-information file may be compressed into zip format. Other encryption formats may also be applied to the serialized assay definition information. For example, the meta-information may include one or more of the following:

Analysis pipeline version,

Reference genome path for the reference genome file location,

Assay unique name—the assay's internal name for checking the unique occurrence in the system,

Docker image name—to be used for launching analysis and installing assay dependent file references,

Any dependency package names needed for analysis pipeline launch.

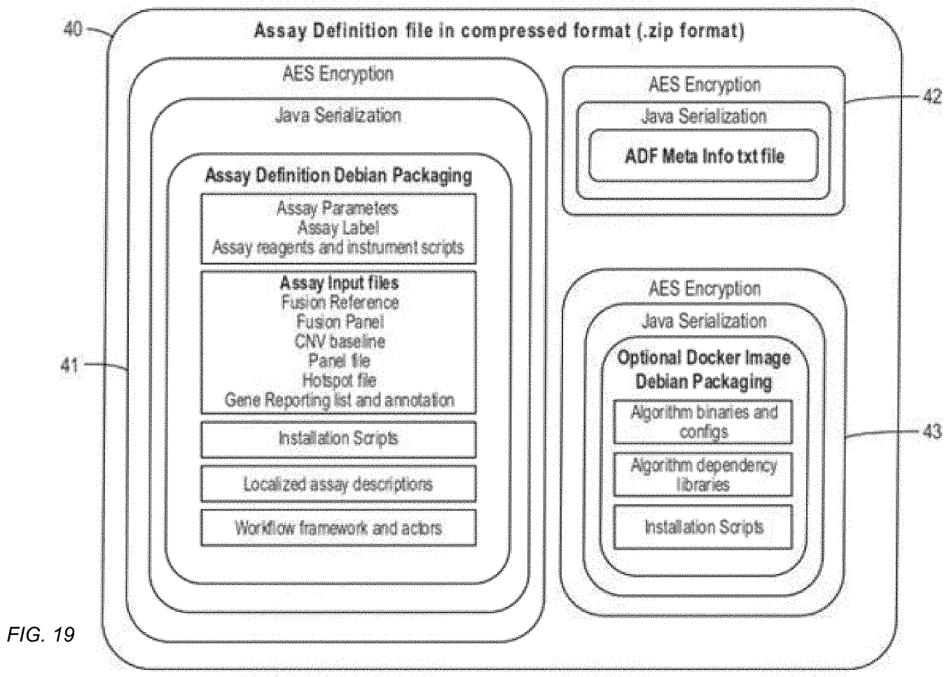

FIG. 19 is a schematic diagram of an example of the assay definition file packaging. The compressed assay definition file in zipped format 40 may include the serialized and encrypted assay definition Debian packaging 41, the serialized and encrypted meta-information text file 42, and serialized and encrypted optional Docker image Debian packaging 43. The server system may decrypt both the meta-information text file 42 and the assay definition serialized file 41 before installing the assay definition Debian file.

The server system and modular software components may be configured to control multiple functional modes, including an RUO, or AD, mode and an IVD, or Dx, mode. Referring to FIG. 16, the Tomcat Server may be configured to include a Web ARchive (WAR) file for the RUO mode and a WAR file for the IVD mode. The server system may be configured to include a RUO variome database for the variants detected by RUO assays and an IVD variome database for the variants detected by IVD assays. The server system may be configured to include separate analysis pipelines and associated Kepler workflow engines for the RUO mode and the IVD mode. The RUO Docker image files for the RUO assays may be configured as separate files from the IVD Docker image files for the IVD assays. The relational databases may be configured to have separate databases: an assay development (AD) database for the RUO mode and a Dx database for the IVD mode. A server system that initially supports only a RUO mode may be configured to support RUO and IVD modes by a software update.

ADFs may be generated separately for RUO mode assays and IVD mode assays. The RUO mode ADFs may include assay definitions for assays used in research. The RUO mode ADFs may be developed by a third party. The IVD mode ADFs include assay definitions for assays compliant with regional regulatory requirements for diagnostic use.

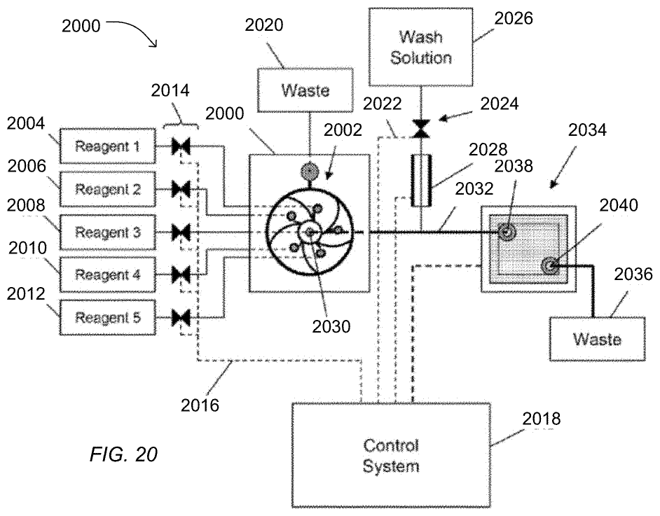

In particular, such methods can be implemented in a sequencing system, such as an optical sequencing system or an ion-based sequencing system. For example, as illustrated in FIG. 20, a system 2000 containing fluidics circuit 2002 is connected by inlets to at least two reagent reservoirs (2004, 2006, 2008, 2010, or 2012), to waste reservoir 2020, and to biosensor 2034 by fluid pathway 2032 that connects fluidics node 2030 to inlet 2038 of biosensor 2034 for fluidic communication. Reagents from reservoirs (2004, 2006, 2008, 2010, or 2012) can be driven to fluidic circuit 2002 by a variety of methods including pressure, pumps, such as syringe pumps, gravity feed, and the like, and are selected by control of valves 2014. Reagents from the fluidics circuit 2002 can be driven through the valves 2014 receiving signals from control system 2018 to waste container 2020. Reagents from the fluidics circuit 2002 can also be driven through the biosensor 2034 to the waste container 2036. The control system 2018 includes controllers for valves 2014, which generate signals for opening and closing via electrical connection 2016.

The control system 2018 also includes controllers for other components of the system, such as wash solution valve 2024 connected thereto by electrical connection 2022, and reference electrode 2028. Control system 2018 can also include control and data acquisition functions for biosensor 2034. In one mode of operation, fluidic circuit 2002 delivers a sequence of selected reagents 1, 2, 3, 4, or 5 to biosensor 2034 under programmed control of control system 2018, such that in between selected reagent flows, fluidics circuit 2002 is primed and washed, and biosensor 2034 is washed. Fluids entering biosensor 2034 exit through outlet 2040 and are deposited in waste container 2036 via control of pinch valve regulator 2044. The valve 2044 is in fluidic communication with the sensor fluid output 2040 of the biosensor 2034.

The device including the dielectric layer defining the well formed from the first access and second access and exposing a sensor pad finds particular use in detecting chemical reactions and byproducts, such as detecting the release of hydrogen ions in response to nucleotide incorporation, useful in genetic sequencing, among other applications. In a particular embodiment, a sequencing system includes a flow cell in which a sensory array is disposed, includes communication circuitry in electronic communication with the sensory array, and includes containers and fluid controls in fluidic communication with the flow cell. In an example, FIG. 21 illustrates an expanded and cross-sectional view of a flow cell 2100 and illustrates a portion of a flow chamber 2106. A reagent flow 2108 flows across a surface of a well array 2102, in which the reagent flow 2108 flows over the open ends of wells of the well array 2102. The well array 2102 and a sensor array 2105 together may form an integrated unit forming a lower wall (or floor) of flow cell 2100. A reference electrode 2104 may be fluidly coupled to flow chamber 2106. Further, a flow cell cover 2130 encapsulates flow chamber 2106 to contain reagent flow 2108 within a confined region.

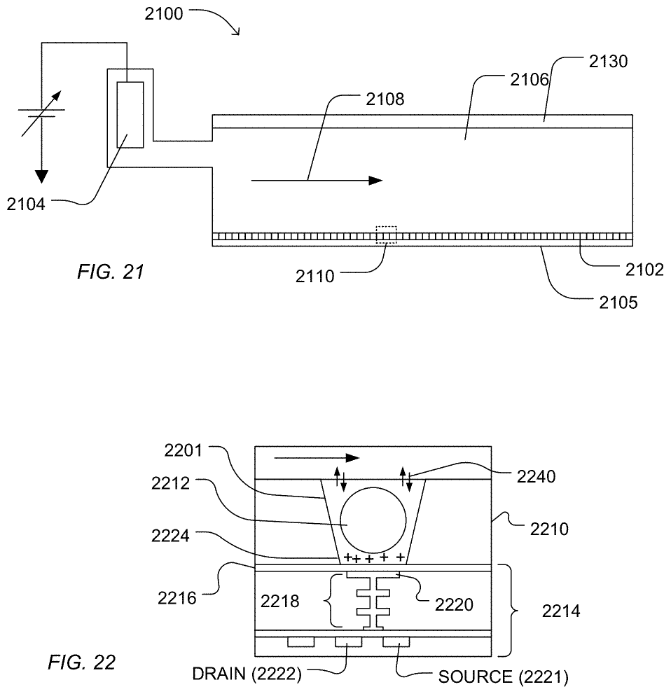

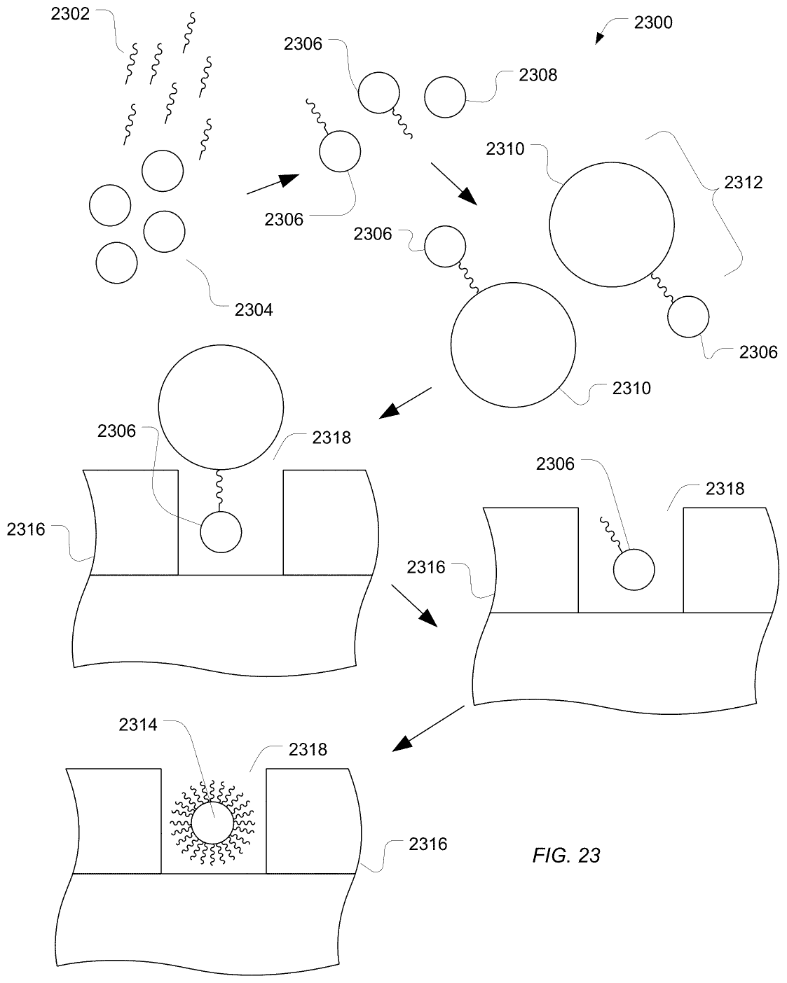

FIG. 22 illustrates an expanded view of a well 2201 and a sensor 2214, as illustrated at 2110 of FIG. 21. The volume, shape, aspect ratio (such as base width-to-well depth ratio), and other dimensional characteristics of the wells may be selected based on the nature of the reaction taking place, as well as the reagents, byproducts, or labeling techniques (if any) that are employed. The sensor 2214 can be a chemical field-effect transistor (chemFET), more specifically an ion-sensitive FET (ISFET), with a floating gate 2218 having a sensor plate 2220 optionally separated from the well interior by a material layer 2216. The sensor 2214 can be responsive to (and generate an output signal related to) the amount of a charge 2224 present on the material layer 2216 opposite the sensor plate 2220. The material layer 2216 can be a ceramic layer, such as an oxide of zirconium, hafnium, tantalum, aluminum, or titanium, among others, or a nitride of titanium. Alternatively, the material layer 2216 can be formed of a metal, such as titanium, tungsten, gold, silver, platinum, aluminum, copper, or a combination thereof. In an example, the material layer 2216 can have a thickness in a range of 5 nm to 100 nm, such as a range of 10 nm to 70 nm, a range of 15 nm to 65 nm, or even a range of 20 nm to 50 nm.

While the material layer 2216 is illustrated as extending beyond the bounds of the illustrated FET component, the material layer 2216 can extend along the bottom of the well 2201 and optionally along the walls of the well 2201. The sensor 2214 can be responsive to (and generate an output signal related to) the amount of a charge 2224 present on the material layer 2216 opposite the sensor plate 2220. Changes in the charge 2224 can cause changes in a current between a source 2221 and a drain 2222 of the chemFET. In turn, the chemFET can be used directly to provide a current-based output signal or indirectly with additional circuitry to provide a voltage-based output signal. Reactants, wash solutions, and other reagents may move in and out of the wells by a diffusion mechanism 2240.

The well 2201 can be defined by a wall structure, which can be formed of one or more layers of material. In an example, the wall structure can have a thickness extending from the lower surface to the upper surface of the well in a range of 0.01 micrometers to 10 micrometers, such as a range of 0.05 micrometers to 10 micrometers, a range of 0.1 micrometers to 10 micrometers, a range of 0.3 micrometers to 10 micrometers, or a range of 0.5 micrometers to 6 micrometers. In particular, the thickness can be in a range of 0.01 micrometers to 1 micrometer, such as a range of 0.05 micrometers to 0.5 micrometers, or a range of 0.05 micrometers to 0.3 micrometers. The wells 301 of array 202 can have a characteristic diameter, defined as the square root of 4 times the cross-sectional area (A) divided by Pi (e.g., sqrt(4*A/π)), of not greater than 5 micrometers, such as not greater than 3.5 micrometers, not greater than 2.0 micrometers, not greater than 1.6 micrometers, not greater than 1.0 micrometers, not greater than 0.8 micrometers or even not greater than 0.6 micrometers. In an example, the wells 301 can have a characteristic diameter of at least 0.01 micrometers. In a further example, the well 301 can define a volume in a range of 0.05 fL to 10 pL, such as a volume in a range of 0.05 fL to 1 pL, a range of 0.05 fL to 100 fL, a range of 0.05 fL to 10 fL, or even a range of 0.1 fL to 5 fL.

In an embodiment, reactions carried out in the well 2201 can be analytical reactions to identify or determine characteristics or properties of an analyte of interest. Such reactions can generate directly or indirectly byproducts that affect the amount of charge adjacent to the sensor plate 2220. If such byproducts are produced in small amounts or rapidly decay or react with other constituents, then multiple copies of the same analyte may be analyzed in the well 2201 at the same time in order to increase the output signal generated. In an embodiment, multiple copies of an analyte may be attached to a solid phase support 2212, either before or after deposition into the well 2201. The solid phase support 2212 may be microparticles, nanoparticles, beads, solid or porous comprising gels, or the like. For simplicity and ease of explanation, solid phase support 2212 is also referred herein as a particle or bead. For a nucleic acid analyte, multiple, connected copies may be made by rolling circle amplification (RCA), exponential RCA, or like techniques, to produce an amplicon without the need of a solid support.

In particular, the solid phase support, such a bead support, can include copies of polynucleotides. In a particular example illustrated in FIG. 23, polymeric particles can be used as a support for polynucleotides during sequencing techniques. For example, such hydrophilic particles can immobilize a polynucleotide for sequencing using fluorescent sequencing techniques. In another example, the hydrophilic particles can immobilize a plurality of copies of a polynucleotide for sequencing using ion-sensing techniques. Alternatively, the above described treatments can improve polymer matrix bonding to a surface of a sensor array. The polymer matrices can capture analytes, such as polynucleotides for sequencing.

A bead support may be composed of organic polymers such as polystyrene, polyethylene, polypropylene, polyfluoroethylene, polyethyleneoxy, and polyacrylamide, as well as co-polymers and grafts thereof. A support may also be inorganic, such as glass, silica, controlled-pore-glass (CPG), or reverse-phase silica. The configuration of a support may be in the form of beads, spheres, particles, granules, a gel, or a surface. Supports may be porous or non-porous, and may have swelling or non-swelling characteristics. In some embodiments, a support is an Ion Sphere Particle. Example bead supports are disclosed in U.S. Pat. No. 9,243,085, titled “Hydrophilic Polymeric Particles and Methods for Making and Using Same,” and in U.S. Pat. No. 9,868,826, titled “Polymer Substrates Formed from Carboxy Functional Acrylamide,” each of which is incorporated herein by reference.

In some embodiments, the solid support is a “microparticle,” “bead,” “microbead,” etc., (optionally but not necessarily spherical in shape) having a smallest cross-sectional length (e.g., diameter) of 50 microns or less, preferably 10 microns or less, 3 microns or less, approximately 1 micron or less, approximately 0.5 microns or less, e.g., approximately 0.1, 0.2, 0.3, or 0.4 microns, or smaller (e.g., under 1 nanometer, about 1-10 nanometer, about 10-100 nanometers, or about 100-500 nanometers). In an example, the support is at least 0.1 microns. Microparticles or bead supports may be made of a variety of inorganic or organic materials including, but not limited to, glass (e.g., controlled pore glass), silica, zirconia, cross-linked polystyrene, polyacrylate, polymethylmethacrylate, titanium dioxide, latex, polystyrene, etc. Magnetization can facilitate collection and concentration of the microparticle-attached reagents (e.g., polynucleotides or ligases) after amplification, and can also facilitate additional steps (e.g., washes, reagent removal, etc.). In certain embodiments, a population of microparticles having different shapes sizes or colors is used. The microparticles can optionally be encoded, e.g., with quantum dots such that each microparticle or group of microparticles can be individually or uniquely identified.

Magnetic beads (e.g., Dynabeads from Dynal, Oslo, Norway) can have a size in a range of 1 micron to 100 microns, such as 2 microns to 100 microns. The magnetic beads can be formed of inorganic or organic materials including, but not limited to, glass (e.g., controlled pore glass), silica, zirconia, cross-linked polystyrene, polystyrene, or a combination thereof.

In some embodiments, a bead support is functionalized for attaching a population of first primers. In some embodiments, a bead is any size that can fit into a reaction chamber. For example, one bead can fit in a reaction chamber. In some embodiments, more than one bead fit in a reaction chamber. In some embodiments, the smallest cross-sectional length of a bead (e.g., diameter) is about 50 microns or less, or about 10 microns or less, or about 3 microns or less, approximately 1 micron or less, approximately 0.5 microns or less, e.g., approximately 0.1, 0.2, 0.3, or 0.4 microns, or smaller (e.g., under 1 nanometer, about 1-10 nanometer, about 10-100 nanometers, or about 100-500 nanometers).

In general, the bead support can be treated to include a biomolecule, including nucleosides, nucleotides, nucleic acids (oligonucleotides and polynucleotides), polypeptides, saccharides, polysaccharides, lipids, or derivatives or analogs thereof. For example, a polymeric particle can bind or attach to a biomolecule. A terminal end or any internal portion of a biomolecule can bind or attach to a polymeric particle. A polymeric particle can bind or attach to a biomolecule using linking chemistries. A linking chemistry includes covalent or non-covalent bonds, including an ionic bond, hydrogen bond, affinity bond, dipole-dipole bond, van der Waals bond, and hydrophobic bond. A linking chemistry includes affinity between binding partners, for example between: an avidin moiety and a biotin moiety; an antigenic epitope and an antibody or immunologically reactive fragment thereof; an antibody and a hapten; a digoxigen moiety and an anti-digoxigen antibody; a fluorescein moiety and an anti-fluorescein antibody; an operator and a repressor; a nuclease and a nucleotide; a lectin and a polysaccharide; a steroid and a steroid-binding protein; an active compound and an active compound receptor; a hormone and a hormone receptor; an enzyme and a substrate; an immunoglobulin and protein A; or an oligonucleotide or polynucleotide and its corresponding complement.

As illustrated in FIG. 23, a plurality of bead supports 2304 can be placed in a solution along with a plurality of polynucleotides 2302 (target or template poylnucleotides). The plurality of bead supports 2304 can be activated or otherwise prepared to bind with the polynucleotides 2302. For example, the bead supports 2304 can include an oligonucleotide (capture primer) complementary to a portion of a polynucleotide of the plurality of polynucleotides 2302. In another example, the bead supports 2304 can be modified with target polynucleotides 2302 using techniques such as biotin-streptavidin binding.

In some embodiments, the template nucleic acid molecules (template polynucleotides or target polynucleotides) can be derived from a sample that can be from a natural or non-natural source. The nucleic acid molecules in the sample can be derived from a living organism or a cell. Any nucleic acid molecule can be used, for example, the sample can include genomic DNA covering a portion of or an entire genome, mRNA, or miRNA from the living organism or cell. In other embodiments, the template nucleic acid molecules can be synthetic or recombinant. In some embodiments, the sample contains nucleic acid molecules having substantially identical sequences or having a mixture of different sequences. Illustrative embodiments are typically performed using nucleic acid molecules that were generated within and by a living cell. Such nucleic acid molecules are typically isolated directly from a natural source such as a cell or a bodily fluid without any in vitro amplification. Accordingly, the sample nucleic acid molecules are used directly in subsequent steps. In some embodiments, the nucleic acid molecules in the sample can include two or more nucleic acid molecules with different sequences.

The methods can optionally include a target enrichment step before, during, or after the library preparation and before a pre-seeding reaction. Target nucleic acid molecules, including target loci or regions of interest, can be enriched, for example, through multiplex nucleic acid amplification or hybridization. A variety of methods can be used to perform multiplex nucleic acid amplification to generate amplicons, such as multiplex PCR, and can be used in an embodiment. Enrichment by any method can be followed by a universal amplification reaction before the template nucleic acid molecules are added to a pre-seeding reaction mixture. Any of the embodiments of the present teachings can include enriching a plurality of at least 2, 3, 4, 5, 6, 7, 8, 9, 10, 15, 20, 25, 30, 35, 40, 45, 50, 55, 60, 65, 70, 75, 80, 85, 90, 95, 100, 125, 150, 175, 200, 250, 300, 400, 500, 600, 700, 800, 900, 1,000, 2,000, 3,000, 4,000, 5,000, 6,000, 7,000, 8,000, 9,000, or 10,000 target nucleic acid molecules, target loci, or regions of interest. In any of the disclosed embodiments, the target loci or regions of interest can be at least 1, 2, 3, 4, 5, 6, 7, 8, 9, 10, 11, 12, 13, 14, 15, 16, 17, 18, 19, 20, 25, 50, 75, 100, 125, 150, 200, 250, 300, 400, 500, 600, 700, 800, 900, or 1,000 nucleotides in length and include a portion of or the entirety of the template nucleic acid molecule. In other embodiments, the target loci or regions of interest can be between about 1 and 10,000 nucleotides in length, for example between about 2 and 5,000 nucleotides, between about 2 and 3,000 nucleotides, or between about 2 and 2,000 nucleotides in length. In any of the embodiments of the present teachings, the multiplex nucleic acid amplification can include generating at least 2, 3, 4, 5, 6, 7, 8, 9, 10, 15, 20, 25, 30, 35, 40, 45, 50, 55, 60, 65, 70, 75, 80, 85, 90, 95, 100, 125, 150, 175, 200, 250, 300, 400, 500, 600, 700, 800, 900, 1,000, 2,000, 3,000, 4,000, 5,000, 6,000, 7,000, 8,000, 9,000, or 10,000 copies of each target nucleic acid molecule, target locus, or region of interest.

In some embodiments, after the library preparation and optional enrichment step, the library of template nucleic acid molecules can be templated onto one or more supports. The one or more supports can be templated in two reactions, a seeding reaction to generate pre-seeded solid supports and a templating reaction using the one or more pre-seeded supports to further amplify the attached template nucleic acid molecules. The pre-seeding reaction is typically an amplification reaction and can be performed using a variety of methods. For example, the pre-seeding reaction can be performed in an RPA reaction, a template walking reaction, or a PCR. In an RPA reaction, template nucleic acid molecules are amplified using a recombinase, polymerase, and optionally a recombinase accessory protein in the presence of primers and nucleotides. The recombinase and optionally the recombinase accessory protein can dissociate at least a portion of a double stranded template nucleic acid molecules to allow primers to hybridize that the polymerase can then bind to initiate replication. In some embodiments, the recombinase accessory protein can be a single-stranded binding protein (SSB) that prevents the re-hybridization of dissociated template nucleic acid molecules. Typically, RPA reactions can be performed at isothermal temperatures. In a template walking reaction, template nucleic acid molecules are amplified using a polymerase in the presence of primers and nucleotides in reaction conditions that allow at least a portion of double-stranded template nucleic acid molecules to dissociate such that primers can hybridize and the polymerase can then bind to initiate replication. In PCR, the double-stranded template nucleic acid molecules are dissociated by thermal cycling. After cooling, primers bind to complementary sequences and can be used for replication by the polymerase. In any of the aspects of the present teachings, the pre-seeding reaction can be performed in a pre-seeding reaction mixture, which is formed with the components necessary for amplification of the template nucleic acid molecules. In any of the disclosed aspects, the pre-seeding reaction mixture can include some or all of the following: a population of template nucleic acid molecules, a polymerase, one or more solid supports with a population of attached first primers, nucleotides, and a cofactor such as a divalent cation. In some embodiments, the pre-seeding reaction mixture can further include a second primer and optionally a diffusion-limiting agent. In some embodiments, the population of template nucleic acid molecules comprise template nucleic acid molecules joined to at least one adaptor sequence which can hybridize to the first or second primers. In some embodiments, the reaction mixture can form an emulsion, as in emulsion RPA or emulsion PCR. In pre-seeding reactions carried out by RPA reactions, the pre-seeding reaction mixture can include a recombinase and optionally a recombinase accessory protein. The various components of the reaction mixture are discussed in further detail herein.

In a particular embodiment of seeding, the hydrophilic particles and polynucleotides are subjected to polymerase chain reaction (PCR) amplification or recombinase polymerase amplification (RPA). In an example, the particles 2304 include a capture primer complementary to a portion of the template polynucleotide 2302. The template polynucleotide can hybridize to the capture primer. The capture primer can be extended to form beads 2306 that include a target polynucleotide attached thereto. Other beads may remain unattached to a target nucleic acid and other template polynucleotide can be free floating in solution.

In an example, the bead support 2306 including a target polynucleotide can be attached to a magnetic bead 2310 to form a bead assembly 2312. In particular, the magnetic bead 2310 is attached to the bead support 2306 by a double stranded polynucleotide linkage. In an example, a further probe including a linker moiety can hybridize to a portion of the target polynucleotide on the bead support 2306. The linker moiety can be attached to a complementary linker moiety on the magnetic bead 2310. In another example, the template polynucleotide used to form the target nucleic acid attached to beads 2306 can include a linker moiety that attaches to the magnetic bead 2310. In another example, the template polynucleotide complementary to target polynucleotide attached to the bead support 2306 can be generated from a primer that is modified with a linker that attaches to the magnetic bead 2310.

The linker moiety attached to the polynucleotide and the linker moiety attached to the magnetic bead can be complementary to and attach to each other. In an example, the linker moieties have affinity and can include: an avidin moiety and a biotin moiety; an antigenic epitope and an antibody or immunologically reactive fragment thereof; an antibody and a hapten; a digoxigen moiety and an anti-digoxigen antibody; a fluorescein moiety and an anti-fluorescein antibody; an operator and a repressor; a nuclease and a nucleotide; a lectin and a polysaccharide; a steroid and a steroid-binding protein; an active compound and an active compound receptor; a hormone and a hormone receptor; an enzyme and a substrate; an immunoglobulin and protein A; or an oligonucleotide or polynucleotide and its corresponding complement. In a particular example, the linker moiety attached to the polynucleotide includes biotin and the linker moiety attached to the magnetic bead includes streptavidin.

The bead assemblies 2312 can be applied over a substrate 2316 of a sequencing device that includes wells 2318. In an example, a magnetic field can be applied to the substrate 2316 to draw the magnetic beads 2310 of the bead assembly 2312 towards the wells 2318. The bead support 2306 enters the well 2318. For example, a magnet can be moved in parallel to a surface of the substrate 2316 resulting in the deposition of the bead support 2306 in the wells 2318.

The bead assembly 2312 can be denatured to remove the magnetic bead 2310 leaving the bead support 2306 in the well 2318. For example, hybridized double-stranded DNA of the bead assembly 2312 can be denatured using thermal cycling or ionic solutions to release the magnetic bead 2310 and template polynucleotides having a linker moiety attached to the magnetic bead 2310. For example, the double-stranded DNA can be treated with low ion-content aqueous solutions, such as deionized water, to denature and separate the strands. In an example, a foam wash can be used to remove the magnetic beads.

Optionally, the target polynucleotides 2306 can be amplified, referred to herein as templating, while in the well 2318, to provide a bead support 2314 with multiple copies of the target polynucleotides. In particular, the bead 2314 has a monoclonal population of target polynucleotides. Such an amplification reactions can be performed using polymerase chain reaction (PCR) amplification, recombination polymerase amplification (RPA) or a combination thereof. Alternatively, amplification can be performed prior to depositing the bead support 2314 in the well.

In a particular embodiment, an enzyme such as a polymerase is present, bound to, or is in close proximity to the particles or beads. In an example, a polymerase is present in solution or in the well to facilitate duplication of the polynucleotide. A variety of nucleic acid polymerase may be used in the methods described herein. In an example embodiment, the polymerase can include an enzyme, fragment, or subunit thereof, which can catalyze duplication of the polynucleotide. In another embodiment, the polymerase can be a naturally occurring polymerase, recombinant polymerase, mutant polymerase, variant polymerase, fusion or otherwise engineered polymerase, chemically modified polymerase, synthetic molecules, or analog, derivative or fragment thereof. Example enzymes, solutions, compositions, and amplification methods can be found in WO2019/094,524, titled “METHODS AND COMPOSITIONS FOR MANIPULATING NUCLEIC ACIDS”, which is incorporated herein by reference in its entirety.

While the polynucleotides of bead support 2314 are illustrated as being on a surface, the polynucleotides can extend within the bead support 2314. Hydrogel and hydrophilic particles having a low concentration of polymer relative to water can include polynucleotide segments on the interior of and throughout the bead support 2314 or polynucleotides can reside in pores and other openings. In particular, the bead support 2314 can permit diffusion of enzymes, nucleotides, primers, and reaction products used to monitor the reaction. A high number of polynucleotides per particle produces a better signal.

In an example embodiment, the bead support 2314 can be utilized in a sequencing device. For example, a sequencing device 2316 can include an array of wells 2318. In an example, a sequencing primer can be added to the wells 2318 or the bead support 2314 can be pre-exposed to the primer prior to placement in the well 2318. In particular, the bead support 2314 can include bound sequencing primer. The sequencing primer and polynucleotide form a nucleic acid duplex including the polynucleotide (e.g., a template nucleic acid) hybridized to the sequencing primer. The nucleic acid duplex is an at least partially double-stranded polynucleotide. Enzymes and nucleotides can be provided to the well 2318 to facilitate detectible reactions, such as nucleotide incorporation.

Sequencing can be performed by detecting nucleotide addition. Nucleotide addition can be detected using methods such as fluorescent emission methods or ion detection methods. For example, a set of fluorescently labeled nucleotides can be provided to the system 2316 and can migrate to the well 2318. Excitation energy can be also provided to the well 2318. When a nucleotide is captured by a polymerase and added to the end of an extending primer, a label of the nucleotide can fluoresce, indicating which type of nucleotide is added.

In an alternative example, solutions including a single type of nucleotide can be fed sequentially. In response to nucleotide addition, the pH within the local environment of the well 2318 can change. Such a change in pH can be detected by ion sensitive field effect transistors (ISFET). As such, a change in pH can be used to generate a signal indicating the order of nucleotides complementary to the polynucleotide of the particle 2310.

In particular, a sequencing system can include a well, or a plurality of wells, disposed over a sensor pad of an ionic sensor, such as a field effect transistor (FET). In embodiments, a system includes one or more polymeric particles loaded into a well which is disposed over a sensor pad of an ionic sensor (e.g., FET), or one or more polymeric particles loaded into a plurality of wells which are disposed over sensor pads of ionic sensors (e.g., FET). In embodiments, an FET can be a chemFET or an ISFET. A “chemFET” or chemical field-effect transistor, includes a type of field effect transistor that acts as a chemical sensor. The chemFET has the structural analog of a MOSFET transistor, where the charge on the gate electrode is applied by a chemical process. An “ISFET” or ion-sensitive field-effect transistor, can be used for measuring ion concentrations in solution; when the ion concentration (such as H+) changes, the current through the transistor changes accordingly.

In embodiments, the FET may be a FET array. As used herein, an “array” is a planar arrangement of elements such as sensors or wells. The array may be one or two dimensional. A one-dimensional array can be an array having one column (or row) of elements in the first dimension and a plurality of columns (or rows) in the second dimension. The number of columns (or rows) in the first and second dimensions may or may not be the same. The FET or array can comprise 10², 10³, 10⁴, 10⁵, 10⁶, 10⁷ or more FETs.

In embodiments, one or more microfluidic structures can be fabricated above the FET sensor array to provide for containment or confinement of a biological or chemical reaction. For example, in one implementation, the microfluidic structure(s) can be configured as one or more wells (or wells, or reaction chambers, or reaction wells, as the terms are used interchangeably herein) disposed above one or more sensors of the array, such that the one or more sensors over which a given well is disposed detect and measure analyte presence, level, or concentration in the given well. In embodiments, there can be a 1:1 correspondence of FET sensors and reaction wells.

Returning to FIG. 23, in another example, a well 2318 of the array of wells can be operatively connected to measuring devices. For example, for fluorescent emission methods, a well 2318 can be operatively coupled to a light detection device. In the case of ionic detection, the lower surface of the well 2318 may be disposed over a sensor pad of an ionic sensor, such as a field effect transistor.

One example system involving sequencing via detection of ionic byproducts of nucleotide incorporation is the Ion Torrent PGM™, Proton™, S5™, or Genexus™ sequencer (Thermo Fisher Scientific), which is an ion-based sequencing system that sequences nucleic acid templates by detecting hydrogen ions produced as a byproduct of nucleotide incorporation. Typically, hydrogen ions are released as byproducts of nucleotide incorporations occurring during template-dependent nucleic acid synthesis by a polymerase. The Ion Torrent PGM™, Proton™, S5™, or Genexus™ sequencer detects the nucleotide incorporations by detecting the hydrogen ion byproducts of the nucleotide incorporations. The Ion Torrent PGM™, Proton™, S5™, or Genexus™ sequencer can include a plurality of template polynucleotides to be sequenced, each template disposed within a respective sequencing reaction well in an array. The wells of the array can each be coupled to at least one ion sensor that can detect the release of H+ ions or changes in solution pH produced as a byproduct of nucleotide incorporation. The ion sensor comprises a field effect transistor (FET) coupled to an ion-sensitive detection layer that can sense the presence of H+ ions or changes in solution pH. The ion sensor can provide output signals indicative of nucleotide incorporation which can be represented as voltage changes whose magnitude correlates with the H+ ion concentration in a respective well or reaction chamber. Different nucleotide types can be flowed serially into the reaction chamber and can be incorporated by the polymerase into an extending primer (or polymerization site) in an order determined by the sequence of the template. Each nucleotide incorporation can be accompanied by the release of H+ ions in the reaction well, along with a concomitant change in the localized pH. The release of H+ ions can be registered by the FET of the sensor, which produces signals indicating the occurrence of the nucleotide incorporation. Nucleotides that are not incorporated during a particular nucleotide flow may not produce signals. The amplitude of the signals from the FET can also be correlated with the number of nucleotides of a particular type incorporated into the extending nucleic acid molecule thereby permitting homopolymer regions to be resolved. Thus, during a run of the sequencer multiple nucleotide flows into the reaction chamber along with incorporation monitoring across a multiplicity of wells or reaction chambers can permit the instrument to resolve the sequence of many nucleic acid templates simultaneously.

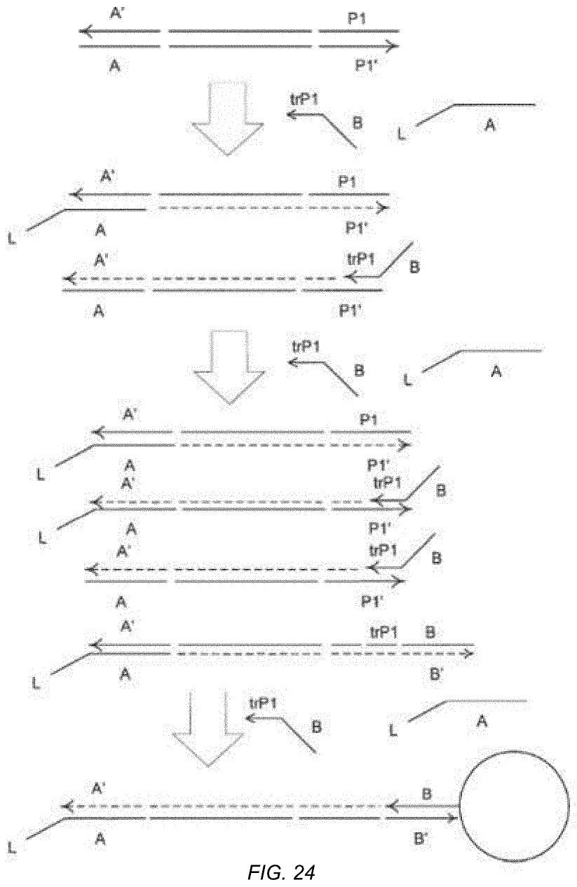

In some embodiments, a pre-seeding (or seeding) reaction can be performed as illustrated in FIG. 24. In this example, a target polynucleotide B-A′ and its complement, a template polynucleotide (A-B′), are amplified in the presence of a bead support having a capture primer. The target polynucleotide has a capture portion (B) the same as or substantially similar to a sequence of the capture primer coupled to the bead support. Substantially similar sequences are sequences whose complements can hybridize to each of the substantially similar sequences. The bead support can have a capture primer that is the same sequence or a sequence substantially similar to that of the B portion of the target polynucleotide to permit hybridization of the complement of the capture portion (B) of the target polynucleotide with the capture primer attached to the bead support. Optionally, the target polynucleotide can include a second primer location (P1) adjacent to the capture portion (B) of the target polynucleotide and can further include a target region adjacent the primers and bounded by complement portion (A′) to a sequencing primer portion (A) of the target polynucleotide. When amplified in the presence of the bead support including a capture primer, the template polynucleotide complementary to the target polynucleotide can hybridize with the capture primer (B). The target polynucleotide can remain in solution. The system can undergo an extension in which the capture primer B is extended complementary to the template polynucleotide yielding a target sequence bound to the bead support. One or more additional amplifications can be performed at this stage in the presence of the support having a capture primer. One or more further amplifications can be performed in the presence of a free primer (B), the bead support, and a free modified sequencing primer (A) a having a linker moiety (L) attached thereto. The primer (B) and the modified primer (L-A) can interfere with the free-floating target polynucleotide and template polynucleotide, hindering them from binding to the bead support and each other. In particular, the modified sequencing primer (A) having the linker moiety attached thereto can hybridize with the complementary portion (A′) of the target polynucleotide attached to the bead support. Optionally, the linker modified sequencing primer L-A hybridized to the target polynucleotide can be extended forming a linker modified template polynucleotide. Such linker modified template polynucleotide hybridized to the target nucleic acid attached to the bead support can then be captured by a magnetic bead and used for magnetic sequestering (enriching) of the target polypeptide attached to the bead and loading of it into a sequencing device. The amplification or extensions can be performed using polymerase chain reaction (PCR) amplification, recombinase polymerase amplification (RPA), or other amplification techniques. In a particular example, each step of the scheme is performed using PCR amplification. Although FIG. 24 depicts a series of reactions for a single double-stranded nucleic acid molecule and a single bead within a single reaction mixture, the same single reaction mixture can contain a plurality of double-stranded nucleic acid molecules and a plurality of beads that are undergoing the same series of reactions to generate at least two beads each attached with one template nucleic acid, or a monoclonal, or substantially monoclonal, population of templates. An example of such system is described in US Published Application No. 2019/0255505A1, which is incorporated by reference in its entirety.

In a first embodiment, a method for determining a sequence of nucleic acids includes extracting nucleic acids from a sample with a purification instrument in accordance with a purification plan of a run plan. The purification plan includes an identifier associated with the source. The method further includes disposing the extracted nucleic acids disposed in a transfer plate following purifying. A well location of the extracted nucleic acids is stored in a transfer file associating the well location on the transfer plate with the identifier. The method also includes transferring the transfer plate to a sequencing instrument; automatically transferring the transfer file to the sequencing instrument; and sequencing at least a portion of the extracted nucleic acids with the sequencing instrument in accordance with a sequencing plan of the run plan and based on the transfer file. The sequencing plan includes the identifier associated with the source and indicates an assay to be used with the extracted nucleic acids associated with the source.

In an example of the first embodiment, a concentration of the extracted nucleic acids is stored in the transfer file.

In another example of the first embodiment and the above examples, the method further includes transferring the transfer file to a server, wherein transferring the transfer file to the sequencing instrument includes transferring the transfer file from the server to the sequencing instrument.

In a further example of the first embodiment and the above examples, the method further includes disposing portions of the extracted nucleic acids in an archive plate. For example, the method further includes storing locations of the portions on the archive plate in the transfer file.

In an additional example of the first embodiment and the above examples, the method further includes preparing the run plan, the run plan including the purification plan and the sequencing plan.

In another example of the first embodiment and the above examples, the purification plan includes an indication of a type of nucleic acids to extract. For example, the purification plan includes an identifier associated with a second source and an indication of a second type of nucleic acids to extract. In an example, the type of nucleic acids and the second type of nucleic acids are different.

In a further example of the first embodiment and the above examples, the sequencing plan includes a reference to an assay definition.

In an additional example of the first embodiment and the above examples, the sequencing plan includes a reference to sequencing chip.

In another example of the first embodiment and the above examples, the sequencing plan associates a nucleic acid tag or barcode with the identifier associated with the host.

In a further example of the first embodiment and the above examples, preparing the run plan includes preparing the run plan on the sequencing instrument. For example, the method further includes storing the run plan with a set of run plans. In an example, the method further includes requesting with the purification instrument the set of run plans and displaying the set of run plans with the purification instrument. In another example, the method further includes receiving a selection of the run plan from a user of the purification instrument.

In an additional example of the first embodiment and the above examples, the method further includes determining automatically with the purification instrument a presence of purification consumables consistent with the purification plan.

In another example of the first embodiment and the above examples, the method further includes determining automatically with the sequencing instrument a presence of sequencing consumables consistent with the sequencing plan.

In a further example of the first embodiment and the above examples, the method further includes providing a purification progress update associated with the run plan from the purification instrument to a server.

In an additional example of the first embodiment and the above examples, the method further includes providing a sequencing progress update associated with the run plan from the sequencer to a server.

In a second embodiment, a system for facilitating sequencing of nucleic acids includes a purification instrument including automation to isolate the nucleic acids from samples and form nucleic acid solutions including the nucleic acids. The purification instrument includes a transfer plate to receive the nucleic acid solutions. The purification instrument is to store plate disposition of the nucleic acid solutions on the transfer plate. The system further includes a sequencer including automation to sequence the nucleic acids. The sequencer is to receive the transfer plate and to sequence the nucleic acids based on the transfer file. The system includes a server to receive the transfer file from the purification instrument and to provide the transfer file to the sequencer. The server is to receive sequence information from the sequencer and to provide sequence analysis.

In an example of the second embodiment, the server includes an FTP service, the purification instrument to provide the transfer file to the server via the FTP service, the sequencer to retrieve the transfer file from the server via the FTP service.

In another example of the second embodiment and the above examples, the server includes a web application server in communication with the purification instrument and the sequencer. For example, the server provides a run plan to the purification instrument via the web application server. In an example, the purification instrument provides progress updates to the server via the web application server. In another example, the server provides a run plan to the sequencer via the web application server.

Note that not all of the activities described above in the general description or the examples are required, that a portion of a specific activity may not be required, and that one or more further activities may be performed in addition to those described. Still further, the order in which activities are listed are not necessarily the order in which they are performed.

In the foregoing specification, the concepts have been described with reference to specific embodiments. However, one of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of invention.

As used herein, the terms “comprises,” “comprising,” “includes,” “including,” “has,” “having” or any other variation thereof, are intended to cover a non-exclusive inclusion. For example, a process, method, article, or apparatus that comprises a list of features is not necessarily limited only to those features but may include other features not expressly listed or inherent to such process, method, article, or apparatus. Further, unless expressly stated to the contrary, “or” refers to an inclusive-or and not to an exclusive-or. For example, a condition A or B is satisfied by any one of the following: A is true (or present) and B is false (or not present), A is false (or not present) and B is true (or present), and both A and B are true (or present).

Also, the use of “a” or “an” are employed to describe elements and components described herein. This is done merely for convenience and to give a general sense of the scope of the invention. This description should be read to include one or at least one and the singular also includes the plural unless it is obvious that it is meant otherwise.

Benefits, other advantages, and solutions to problems have been described above with regard to specific embodiments. However, the benefits, advantages, solutions to problems, and any feature(s) that may cause any benefit, advantage, or solution to occur or become more pronounced are not to be construed as a critical, required, or essential feature of any or all the claims.

After reading the specification, skilled artisans will appreciate that certain features are, for clarity, described herein in the context of separate embodiments, may also be provided in combination in a single embodiment. Conversely, various features that are, for brevity, described in the context of a single embodiment, may also be provided separately or in any subcombination. Further, references to values stated in ranges include each and every value within that range.

What is claimed is:

1. A method for determining a sequence of nucleic acids, the method comprising:
extracting nucleic acids from a sample with a purification instrument in accordance with a purification plan of a run plan, the purification plan including an identifier associated with the source;
disposing the extracted nucleic acids disposed in a transfer plate following purifying, a well location of the extracted nucleic acids being stored in a transfer file associating the well location on the transfer plate with the identifier;
transferring the transfer plate to a sequencing instrument;
automatically transferring the transfer file to the sequencing instrument; and
sequencing at least a portion of the extracted nucleic acids with the sequencing instrument in accordance with a sequencing plan of the run plan and based on the transfer file, the sequencing plan including the identifier associated with the source and indicating an assay to be used with the extracted nucleic acids associated with the source.

2. The method of claim 1, wherein a concentration of the extracted nucleic acids is stored in the transfer file.

3. The method of claim 1, further comprising transferring the transfer file to a server, wherein transferring the transfer file to the sequencing instrument includes transferring the transfer file from the server to the sequencing instrument.

4. The method of claim 1, further comprising disposing portions of the extracted nucleic acids in an archive plate.

5. The method of claim 4, further comprising storing locations of the portions on the archive plate in the transfer file.

6. The method of claim 1, further comprising preparing the run plan, the run plan including the purification plan and the sequencing plan.

7. The method of claim 1, wherein the purification plan includes an indication of a type of nucleic acids to extract.

8. The method of claim 7, wherein the purification plan includes an identifier associated with a second source and an indication of a second type of nucleic acids to extract.

9. The method of claim 8, wherein the type of nucleic acids and the second type of nucleic acids are different.

10. The method of claim 1, wherein the sequencing plan includes a reference to an assay definition.

11. The method of claim 1, wherein the sequencing plan includes a reference to sequencing chip.

12. The method of claim 1, wherein the sequencing plan associates a nucleic acid tag or barcode with the identifier associated with the host.

13. The method of claim 1, wherein preparing the run plan includes preparing the run plan on the sequencing instrument.

14. The method of claim 13, further comprising storing the run plan with a set of run plans.

15. The method of claim 14, further comprising requesting with the purification instrument the set of run plans and displaying the set of run plans with the purification instrument.

16. The method of claim 15, further comprising receiving a selection of the run plan from a user of the purification instrument.

17. The method of claim 1, further comprising determining automatically with the purification instrument a presence of purification consumables consistent with the purification plan.

18. The method of claim 1, further comprising determining automatically with the sequencing instrument a presence of sequencing consumables consistent with the sequencing plan.

19. The method of claim 1, further comprising providing a purification progress update associated with the run plan from the purification instrument to a server.

20. The method of claim 1, further comprising providing a sequencing progress update associated with the run plan from the sequencer to a server.
